(19) United States
(12) Patent Application Publication
(10) Pub. No.: US 2022/0313611 A1
(43) Pub. Date: Oct. 6, 2022

(54) METHODS TO PREPARE DRY POWDERS USING SUSPENSION BASED THIN FILM FREEZING

(71) Applicants: BOARD OF REGENTS, THE UNIVERSITY OF TEXAS SYSTEM, Austin, TX (US); TFF Pharmaceuticals, Inc., Austin, TX (US)

(72) Inventors: Robert O. WILLIAMS, III, Austin, TX (US); Sawittree SAHAKIJPIJARN, Austin, TX (US); Chaeho MOON, Austin, TX (US); John J. KOLENG, JR., Austin, TX (US)

(73) Assignees: BOARD OF REGENTS, THE UNIVERSITY OF TEXAS SYSTEM, Austin, TX (US); TFF Pharmaceuticals, Inc., Austin, TX (US)

(21) Appl. No.: 17/693,062

(22) Filed: Mar. 11, 2022

Related U.S. Application Data
(60) Provisional application No. 63/160,588, filed on Mar. 12, 2021.

(51) Int. Cl. A61K 9/14; A61P 37/02; A61P 31/10; A61P 33/10; A61K 9/16; A61K 9/50; A61K 31/436; A61K 31/506; A61K 31/167

(52) U.S. Cl. CPC A61K 9/141; A61P 37/02; A61P 31/10; A61P 33/10; A61K 9/1694; A61K 9/5073; A61K 31/436; A61K 31/506; A61K 31/167

(57) ABSTRACT

In some aspects, the present disclosure provides methods of preparing pharmaceutical compositions using an suspension based thin film freezing method to obtain inhalable compositions. These compositions may have a higher homogeneity compared to compositions prepared using conventional methods. These compositions may be used to treat or prevent one or more diseases or disorders.

This application claims the benefit of priority to U.S. Provisional Application No. 63/160,588, filed on Mar. 12, 2021, the entire contents of which are hereby incorporated by reference.

### BACKGROUND

### 1. Field

The present disclosure relates generally to the field of pharmaceuticals and pharmaceutical manufacture. More particularly, it concerns methods of preparing a pharmaceutical composition comprising suspensions of drug particles to make dry powders.

### 2. Description of Related Art

Pulmonary drug delivery has advanced significantly over the last decade. Orally inhaled products have been developed as delivery systems for both local treatments of lung diseases (e.g., chronic obstructive pulmonary disease, asthma, tuberculosis) and the systemic treatment of several diseases such as diabetes (Pfutzner and Forst, 2005), measles (Griffin, 2014), Parkinson's disease (LeWitt et al., 2018), schizophrenia (Kristin et al., 2016), and influenza (Silveira et al., 2016). The dry powder inhaler (DPI) is considered the most promising dosage form, as opposed to pressurized metered-dose inhalers or nebulizers. DPIs provide several advantages, including ease of operation and portability. In addition, they do not require propellants, they allow for relatively low-cost devices, and they offer enhanced stability of the active component as a result of their dry state (Carpenter et al., 1997).

The development of inhaled products must address several physical difficulties to achieve effective drug delivery. The aerodynamic diameter of drug particles must be between 1 μm and 5 μm to maximize the probability of drug particles from a DPI reaching the lower respiratory tract (Prime et al., 1997). However, such micronized drug particles have high forces of cohesiveness and a tendency to agglomerate, which results in poor flowability, poor aerosolization properties, and high dose variability (Chan and Chew, 2003). As such, there is an unmet need for methods of preparing inhalable pharmaceutical compositions with improved properties.

### SUMMARY OF THE DISCLOSURE

In some aspects, the present disclosure provides methods of preparing a pharmaceutical composition comprising:
(A) obtaining a solution of an active pharmaceutical ingredient in a solvent;
(B) adding a carrier to the mixture to obtain a dispersion;
(C) depositing the dispersion onto a surface;
(D) subjecting the dispersion to a reduced temperature such that the dispersion freezes to obtain a frozen dispersion; and
(E) subjecting the frozen dispersion to a drying process to obtain a pharmaceutical composition;

wherein the pharmaceutical composition contains one or more particles wherein the active pharmaceutical ingredient has been deposited on the surface of the carrier and the pharmaceutical composition comprises both the active pharmaceutical ingredient and the carrier in a single particle.

In some embodiments, the dispersion further comprises a further excipient. In some embodiments, the excipient is an amino acid such as a hydrophobic amino acid. In some embodiments, the amino acid is leucine or trileucine. In some embodiments, the pharmaceutical composition comprises from about 0.05% w/w to about 50% w/w of the excipient. In some embodiments, the pharmaceutical composition comprises from about 1% w/w to about 15% w/w of the excipient. In some embodiments, the pharmaceutical composition comprises from about 2.5% w/w to about 10% w/w of the excipient. In some embodiments, the carrier is a sugar or sugar alcohol such as a polysaccharide. In some embodiments, the polysaccharide is lactose.

In some embodiments, the carrier is sparingly soluble in the solvent. In some embodiments, the carrier is slightly soluble. In some embodiments, the carrier is very slightly soluble. In some embodiments, the carrier is practically insoluble. In some embodiments, the dispersion is a suspension.

In some embodiments, the pharmaceutical composition comprises at least 60% of the carrier that is in the amorphous form. In some embodiments, the pharmaceutical composition comprises at least 80% of the carrier that is in the amorphous form. In some embodiments, the pharmaceutical composition comprises at least 90% of the carrier that is in the amorphous form. In some embodiments, the pharmaceutical composition comprises at least 95% of the carrier that is in the amorphous form. In some embodiments, the pharmaceutical composition comprises at least 98% of the carrier that is in the amorphous form. In some embodiments, the pharmaceutical composition comprises at least 99% of the carrier that is in the amorphous form. In some embodiments, the pharmaceutical composition comprises at least 60% of the carrier that is in the crystalline form. In some embodiments, the pharmaceutical composition comprises at least 80% of the carrier that is in the crystalline form. In some embodiments, the pharmaceutical composition comprises at least 90% of the carrier that is in the crystalline form. In some embodiments, the pharmaceutical composition comprises at least 95% of the carrier that is in the crystalline form. In some embodiments, the pharmaceutical composition comprises at least 98% of the carrier that is in the crystalline form. In some embodiments, the pharmaceutical composition comprises at least 99% of the carrier that is in the crystalline form. In some embodiments, the pharmaceutical composition comprises from about 50% w/w to about 99% w/w of the carrier. In some embodiments, the pharmaceutical composition comprises from about 60% w/w to about 95% w/w of the carrier. In some embodiments, the pharmaceutical composition comprises from about 65% w/w to about 90% w/w of the carrier.

In some embodiments, the mixture further comprises a pharmaceutically acceptable polymer. In some embodiments, the pharmaceutically acceptable polymer is a non-cellulosic non-ionizable polymer. In some embodiments, the non-cellulosic non-ionizable polymer is a polyvinylpyrrolidone. In some embodiments, the pharmaceutically acceptable polymer has a molecular weight from about 5,000 to about 100,000. In some embodiments, the molecular weight is from about 10,000 to about 50,000. In some embodiments, the molecular weight is from about 20,000 to about 30,000. In some embodiments, the pharmaceutical composition comprises from about 0.5% w/w to about 20% w/w of the pharmaceutically acceptable polymer. In some embodiments, the pharmaceutical composition comprises from about 1% w/w to about 15% w/w of the pharmaceutically acceptable polymer. In some embodiments, the pharmaceutical composition comprises from about 2.5% w/w to about 10% w/w of the pharmaceutically acceptable polymer.

In some embodiments, the solvent is an organic solvent. In some embodiments, the organic solvent is a polar aprotic solvent. In some embodiments, the organic solvent is acetonitrile, tert-butanol, or 1,4-dioxane. In some embodiments, the solvent is 1,4-dioxane or acetonitrile. In some embodiments, the solvent is a mixture of 1,4-dioxane and acetonitrile. In some embodiments, the solvent is a mixture of t-butanol and acetonitrile.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the active pharmaceutical ingredient is antifungal agent. In some embodiments, the antifungal agent is an azole antifungal agent such as voriconazole. In other embodiments, the active pharmaceutical ingredient is immunomodulating drug. In some embodiments, the immunomodulating drug is an immunosuppressing drug such as tacrolimus. In some embodiments, the active pharmaceutical ingredient is anthelmintic agent such as niclosamide.

In some embodiments, the pharmaceutical composition comprises at least 60% of the active pharmaceutical ingredient that is in the amorphous form. In some embodiments, the pharmaceutical composition comprises at least 80% of the active pharmaceutical ingredient that is in the amorphous form. In some embodiments, the pharmaceutical composition comprises at least 90% of the active pharmaceutical ingredient that is in the amorphous form. In some embodiments, the pharmaceutical composition comprises at least 95% of the active pharmaceutical ingredient that is in the amorphous form. In some embodiments, the pharmaceutical composition comprises at least 98% of the active pharmaceutical ingredient that is in the amorphous form. In some embodiments, the pharmaceutical composition comprises at least 99% of the active pharmaceutical ingredient that is in the amorphous form. In some embodiments, the pharmaceutical composition comprises at least 60% of the active pharmaceutical ingredient that is in the crystalline form. In some embodiments, the pharmaceutical composition comprises at least 80% of the active pharmaceutical ingredient that is in the crystalline form. In some embodiments, the pharmaceutical composition comprises at least 90% of the active pharmaceutical ingredient that is in the crystalline form. In some embodiments, the pharmaceutical composition comprises at least 95% of the active pharmaceutical ingredient that is in the crystalline form. In some embodiments, the pharmaceutical composition comprises at least 98% of the active pharmaceutical ingredient that is in the crystalline form. In some embodiments, the pharmaceutical composition comprises at least 99% of the active pharmaceutical ingredient that is in the crystalline form. In some embodiments, the pharmaceutical composition comprises from about 1% w/w to about 50% w/w of the active pharmaceutical ingredient. In some embodiments, the pharmaceutical composition comprises from about 2.5% w/w to about 40% w/w of the active pharmaceutical ingredient. In some embodiments, the pharmaceutical composition comprises from about 5% w/w to about 35% w/w of the active pharmaceutical ingredient.

In some embodiments, the method further comprises using a surface that has been cooled to a first reduced temperature. In some embodiments, the first reduced temperature is from about 25° C. to about −190° C. In some embodiments, the first reduced temperature is from about −20° C. to about −120° C. In some embodiments, the first reduced temperature is from about from about −60° C. to about −90° C. In some embodiments, the surface rotates at a speed. In some embodiments, the speed is from about 5 rpm to about 500 rpm. In some embodiments, the speed is from about 50 rpm to about 250 rpm. In some embodiments, the speed is from about 50 rpm to about 150 rpm.

In some embodiments, the dispersion is deposited on the surface from a height from about 1 cm to about 250 cm. In some embodiments, the height is from about 2.5 cm to about 100 cm. In some embodiments, the height is from about 5 cm to about 50 cm.

In some embodiments, the dry process comprises lyophilization. In some embodiments, the drying process comprises two drying cycles. In some embodiments, the first drying cycle comprises drying at a first temperature from about 0° C. to about −120° C. In some embodiments, the first temperature is a temperature from about −10° C. to about −80° C. In some embodiments, the first temperature is a temperature from about −20° C. to about −60° C. In some embodiments, the first drying cycle comprises drying at a reduced pressure. In some embodiments, the reduced pressure is a first pressure from about 10 mTorr to about 500 mTorr. In some embodiments, the first pressure is from about 25 mTorr to about 250 mTorr. In some embodiments, the first pressure is from about 50 mTorr to about 150 mTorr.

In some embodiments, the second drying cycle comprises drying at a second temperature from about 0° C. to about 80° C. In some embodiments, the second temperature is a temperature from about 10° C. to about 60° C. In some embodiments, the second temperature is a temperature from about 20° C. to about 50° C. In some embodiments, the second drying cycle comprises drying at a reduced pressure. In some embodiments, the reduced pressure is a second pressure from about 10 mTorr to about 500 mTorr. In some embodiments, the second pressure is from about 25 mTorr to about 250 mTorr. In some embodiments, the second pressure is from about 50 mTorr to about 150 mTorr.

In some embodiments, the carrier has a D₅₀ particle size distribution measured by laser diffractometer from about 0.1 μm to about 20 μm. In some embodiments, the D₅₀ particle size distribution is from about 0.5 μm to about 15 μm. In some embodiments, the D₅₀ particle size distribution is from about 1 μm to about 10 μm. In some embodiments, the carrier has a D₅₀ particle size distribution measured by laser diffractometer from about 30 μm to about 150 μm. In some embodiments, the D₅₀ particle size distribution is from about 40 μm to about 125 μm. In some embodiments, the D₅₀ particle size distribution is from about 70 μm to about 100 μm. In some embodiments, the D₅₀ particle size distribution is from about 40 μm to about 70 μm.

In some embodiments, the pharmaceutical composition comprises one or more particles of the active pharmaceutical ingredient and the carrier are agglomerated. In some embodiments, the pharmaceutical composition comprises particles exhibiting two different forms. In some embodiments, the first form is one or more particles of the active pharmaceutical ingredient and the carrier are agglomerated. In some embodiments, the second form is one or more carrier particles which comprise one or more discrete domains of the active pharmaceutical ingredient deposited on the surface of the carrier. In some embodiments, the active pharmaceutical ingredient in the discrete domains is present as a nanostructured aggregate.

In some embodiments, the pharmaceutical composition has a specific surface area of greater than 2 m²/g. In some embodiments, the specific surface area is from about 2 m²/g to about 100 m²/g. In some embodiments, the specific surface area is from about 2.5 m²/g to about 50 m²/g. In some embodiments, the specific surface area is from about 2.5 m²/g to about 25 m²/g. In some embodiments, the specific surface area is from about 2.5 m²/g to about 10 m²/g. In some embodiments, the pharmaceutical composition has a specific surface area that is 50% greater than the specific surface area of the carrier. In some embodiments, the pharmaceutical composition has a specific surface area that is 75% greater than the specific surface area of the carrier. In some embodiments, the pharmaceutical composition has a specific surface area that is 100% greater than the specific surface area of the carrier.

In some embodiments, the pharmaceutical composition has a mass median aerodynamic diameter (MMAD) from about 1.0 μm to about 10.0 μm. In some embodiments, the MMAD is from about 1.5 μm to about 8.0 μm. In some embodiments, the MMAD is from about 2.0 μm to about 6.0 μm. In some embodiments, the MMAD of the pharmaceutical composition is 10% less than the MMAD of an identical composition prepared using another method. In some embodiments, the MMAD of the pharmaceutical composition is 25% less. In some embodiments, the MMAD of the pharmaceutical composition is 50% less. In some embodiments, the MMAD of the pharmaceutical composition is 100% less.

In some embodiments, the pharmaceutical composition has a geometric standard deviation (GSD) from about 1.0 to about 10.0. In some embodiments, the GSD is from about 1.25 to about 8.0. In some embodiments, the GSD is from about 1.5 to about 6.0.

In some embodiments, the pharmaceutical composition has a fine powder fraction of the recovered dose that is 10% greater than the fine powder fraction of the recovered dose of a pharmaceutical composition prepared according to any other method. In some embodiments, the fine powder fraction of the recovered dose of the pharmaceutical composition is 15% greater. In some embodiments, the fine powder fraction of the recovered dose of the pharmaceutical composition is 20% greater. In some embodiments, the fine powder fraction of the recovered dose of the pharmaceutical composition is 25% greater. In some embodiments, the pharmaceutical composition has a fine powder fraction of the recovered dose of greater than 30%. In some embodiments, the fine powder fraction of the recovered dose is greater than 40%. In some embodiments, the fine powder fraction of the recovered dose is greater than 50%.

In some embodiments, the pharmaceutical composition has an emitted dose of the recovered dose of greater than 70%. In some embodiments, the emitted dose of the recovered dose is greater than 80%. In some embodiments, the emitted dose of the recovered dose is greater than 90%.

In some embodiments, the pharmaceutical composition has a relative standard deviation (RSD) of the homogeneity of the pharmaceutical composition is less than 8%. In some embodiments, the relative standard deviation of the homogeneity of less than 6%. In some embodiments, the relative standard deviation of the homogeneity of less than 4%. In some embodiments, the relative standard deviation of the homogeneity of the pharmaceutical composition is 50% less than the relative standard deviation of the homogeneity of a pharmaceutical composition prepared using other means. In some embodiments, the relative standard deviation of the homogeneity of the pharmaceutical composition is 100% less. In some embodiments, the relative standard deviation of the homogeneity of the pharmaceutical composition is 150% less. In some embodiments, the relative standard deviation of the homogeneity of the pharmaceutical composition is 200% less. In some embodiments, the pharmaceutical composition has a homogeneity from about 95% to about 105%. In some embodiments, the homogeneity is from about 97% to about 103%. In some embodiments, the homogeneity is from about 98% to about 102%. In some embodiments, the relative standard deviation (RSD) of the homogeneity of the pharmaceutical composition is less than 5%. In some embodiments, the relative standard deviation (RSD) of the homogeneity is less than 3%. In some embodiments, the relative standard deviation (RSD) of the homogeneity is less than 1%.

In some embodiments, the pharmaceutical composition has a critical primary pressure that is greater than 10% of an identical pharmaceutical composition prepared by jet milling. In some embodiments, the critical primary pressure is greater than 25%. In some embodiments, the critical primary pressure is greater than 50%.

In some embodiments, the carrier has a Carr's Index of less than 25%. In some embodiments, the Carr's index is less than 20%. In some embodiments, the Carr's index is less than 15%. In some embodiments, the carrier has a tapped density of greater than 250 g/L. In some embodiments, the tapped density is greater than 400 g/L. In some embodiments, the tapped density is greater than 500 g/L. In some embodiments, the carrier has a tapped density from about 250 g/L to about 1500 g/L. In some embodiments, the tapped density is from about 400 g/L to about 1250 g/L. In some embodiments, the tapped density is from about 500 g/L to about 1000 g/L. In some embodiments, the carrier has a poured density of greater than 100 g/L. In some embodiments, the poured density is greater than 150 g/L. In some embodiments, the poured density is greater than 250 g/L. In some embodiments, the carrier has a poured density from about 100 g/L to about 1500 g/L. In some embodiments, the poured density is from about 200 g/L to about 1250 g/L. In some embodiments, the poured density is from about 250 g/L to about 1000 g/L.

In another aspect, the present disclosure proves pharmaceutical compositions prepared described herein.

In still yet another aspect, the present disclosure provides pharmaceutical composition comprising:

(A) an active pharmaceutical ingredient;

(B) a carrier;

wherein the pharmaceutical composition contains one or more particles wherein the active pharmaceutical ingredient has been deposited on the surface of the carrier, the pharmaceutical composition comprises both the active pharmaceutical ingredient and the carrier in a single particle, and the pharmaceutical composition has a specific surface area that is 50% greater than the specific surface area of the carrier.

In some embodiments, the dispersion further comprises a further excipient. In some embodiments, the excipient is an amino acid such as a hydrophobic amino acid. In some embodiments, the amino acid is leucine or trileucine. In some embodiments, the pharmaceutical composition comprises from about 0.05% w/w to about 50% w/w of the excipient. In some embodiments, the pharmaceutical composition comprises from about 1% w/w to about 15% w/w of the excipient. In some embodiments, the pharmaceutical composition comprises from about 2.5% w/w to about 10% w/w of the excipient. In some embodiments, the carrier is a sugar or sugar alcohol such as a polysaccharide. In some embodiments, the polysaccharide is lactose.

In some embodiments, the pharmaceutical composition comprises at least 60% of the carrier that is in the amorphous form. In some embodiments, the pharmaceutical composition comprises at least 80% of the carrier that is in the amorphous form. In some embodiments, the pharmaceutical composition comprises at least 90% of the carrier that is in the amorphous form. In some embodiments, the pharmaceutical composition comprises at least 95% of the carrier that is in the amorphous form. In some embodiments, the pharmaceutical composition comprises at least 98% of the carrier that is in the amorphous form. In some embodiments, the pharmaceutical composition comprises at least 99% of the carrier that is in the amorphous form. In some embodiments, the pharmaceutical composition comprises at least 60% of the carrier that is in the crystalline form. In some embodiments, the pharmaceutical composition comprises at least 80% of the carrier that is in the crystalline form. In some embodiments, the pharmaceutical composition comprises at least 90% of the carrier that is in the crystalline form. In some embodiments, the pharmaceutical composition comprises at least 95% of the carrier that is in the crystalline form. In some embodiments, the pharmaceutical composition comprises at least 98% of the carrier that is in the crystalline form. In some embodiments, the pharmaceutical composition comprises at least 99% of the carrier that is in the crystalline form. In some embodiments, the pharmaceutical composition comprises from about 50% w/w to about 99% w/w of the carrier. In some embodiments, the pharmaceutical composition comprises from about 60% w/w to about 95% w/w of the carrier. In some embodiments, the pharmaceutical composition comprises from about 65% w/w to about 90% w/w of the carrier.

In some embodiments, the mixture further comprises a pharmaceutically acceptable polymer. In some embodiments, the pharmaceutically acceptable polymer is a non-cellulosic non-ionizable polymer. In some embodiments, the non-cellulosic non-ionizable polymer is a polyvinylpyrrolidone. In some embodiments, the pharmaceutically acceptable polymer has a molecular weight from about 5,000 to about 100,000. In some embodiments, the molecular weight is from about 10,000 to about 50,000. In some embodiments, the molecular weight is from about 20,000 to about 30,000. In some embodiments, the pharmaceutical composition comprises from about 0.5% w/w to about 20% w/w of the pharmaceutically acceptable polymer. In some embodiments, the pharmaceutical composition comprises from about 1% w/w to about 15% w/w of the pharmaceutically acceptable polymer. In some embodiments, the pharmaceutical composition comprises from about 2.5% w/w to about 10% w/w of the pharmaceutically acceptable polymer.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the active pharmaceutical ingredient is antifungal agent. In some embodiments, the antifungal agent is an azole antifungal agent such as voriconazole. In other embodiments, the active pharmaceutical ingredient is immunomodulating drug. In some embodiments, the immunomodulating drug is an immunosuppressing drug such as tacrolimus. In some embodiments, the active pharmaceutical ingredient is anthelmintic agent such as niclosamide.

In some embodiments, the pharmaceutical composition comprises at least 60% of the active pharmaceutical ingredient that is in the amorphous form. In some embodiments, the pharmaceutical composition comprises at least 80% of the active pharmaceutical ingredient that is in the amorphous form. In some embodiments, the pharmaceutical composition comprises at least 90% of the active pharmaceutical ingredient that is in the amorphous form. In some embodiments, the pharmaceutical composition comprises at least 95% of the active pharmaceutical ingredient that is in the amorphous form. In some embodiments, the pharmaceutical composition comprises at least 98% of the active pharmaceutical ingredient that is in the amorphous form. In some embodiments, the pharmaceutical composition comprises at least 99% of the active pharmaceutical ingredient that is in the amorphous form. In some embodiments, the pharmaceutical composition comprises at least 60% of the active pharmaceutical ingredient that is in the crystalline form. In some embodiments, the pharmaceutical composition comprises at least 80% of the active pharmaceutical ingredient that is in the crystalline form. In some embodiments, the pharmaceutical composition comprises at least 90% of the active pharmaceutical ingredient that is in the crystalline form. In some embodiments, the pharmaceutical composition comprises at least 95% of the active pharmaceutical ingredient that is in the crystalline form. In some embodiments, the pharmaceutical composition comprises at least 98% of the active pharmaceutical ingredient that is in the crystalline form. In some embodiments, the pharmaceutical composition comprises at least 99% of the active pharmaceutical ingredient that is in the crystalline form. In some embodiments, the pharmaceutical composition comprises from about 1% w/w to about 50% w/w of the active pharmaceutical ingredient. In some embodiments, the pharmaceutical composition comprises from about 2.5% w/w to about 40% w/w of the active pharmaceutical ingredient. In some embodiments, the pharmaceutical composition comprises from about 5% w/w to about 35% w/w of the active pharmaceutical ingredient.

In some embodiments, the carrier has a D₅₀ particle size distribution measured by laser diffractometer from about 0.1 μm to about 20 μm. In some embodiments, the D₅₀ particle size distribution is from about 0.5 μm to about 15 μm. In some embodiments, the D₅₀ particle size distribution is from about 1 μm to about 10 μm. In some embodiments, the carrier has a D₅₀ particle size distribution measured by laser diffractometer from about 30 μm to about 150 μm. In some embodiments, the D₅₀ particle size distribution is from about 40 μm to about 125 μm. In some embodiments, the D₅₀ particle size distribution is from about 70 μm to about 100 μm. In some embodiments, the D₅₀ particle size distribution is from about 40 μm to about 70 μm.

In some embodiments, the pharmaceutical composition comprises one or more particles of the active pharmaceutical ingredient and the carrier are agglomerated. In some embodiments, the pharmaceutical composition comprises particles exhibiting two different forms. In some embodiments, the first form is one or more particles of the active pharmaceutical ingredient and the carrier are agglomerated. In some embodiments, the second form is one or more carrier particles which comprise one or more discrete domains of the active pharmaceutical ingredient deposited on the surface of the carrier. In some embodiments, the active pharmaceutical ingredient in the discrete domains is present as a nanostructured aggregate.

In some embodiments, the pharmaceutical composition has a specific surface area of greater than 2 m²/g. In some embodiments, the specific surface area is from about 2 m²/g to about 100 m²/g. In some embodiments, the specific surface area is from about 2.5 m²/g to about 50 m²/g. In some embodiments, the specific surface area is from about 2.5 m²/g to about 25 m²/g. In some embodiments, the specific surface area is from about 2.5 m²/g to about 10 m²/g. In some embodiments, the pharmaceutical composition has a specific surface area that is 50% greater than the specific surface area of the carrier. In some embodiments, the pharmaceutical composition has a specific surface area that is 75% greater than the specific surface area of the carrier. In some embodiments, the pharmaceutical composition has a specific surface area that is 100% greater than the specific surface area of the carrier.

In some embodiments, the pharmaceutical composition has a mass median aerodynamic diameter (MMAD) from about 1.0 μm to about 10.0 μm. In some embodiments, the MMAD is from about 1.5 μm to about 8.0 μm. In some embodiments, the MMAD is from about 2.0 μm to about 6.0 μm. In some embodiments, the MMAD of the pharmaceutical composition is 10% less than the MMAD of an identical composition prepared using another method. In some embodiments, the MMAD of the pharmaceutical composition is 25% less. In some embodiments, the MMAD of the pharmaceutical composition is 50% less. In some embodiments, the MMAD of the pharmaceutical composition is 100% less.

In some embodiments, the pharmaceutical composition has a geometric standard deviation (GSD) from about 1.0 to about 10.0. In some embodiments, the GSD is from about 1.25 to about 8.0. In some embodiments, the GSD is from about 1.5 to about 6.0.

In some embodiments, the pharmaceutical composition has a fine powder fraction of the recovered dose that is 10% greater than the fine powder fraction of the recovered dose of a pharmaceutical composition prepared according to any other method. In some embodiments, the fine powder fraction of the recovered dose of the pharmaceutical composition is 15% greater. In some embodiments, the fine powder fraction of the recovered dose of the pharmaceutical composition is 20% greater. In some embodiments, the fine powder fraction of the recovered dose of the pharmaceutical composition is 25% greater. In some embodiments, the pharmaceutical composition has a fine powder fraction of the recovered dose of greater than 30%. In some embodiments, the fine powder fraction of the recovered dose is greater than 40%. In some embodiments, the fine powder fraction of the recovered dose is greater than 50%.

In some embodiments, the pharmaceutical composition has an emitted dose of the recovered dose of greater than 70%. In some embodiments, the emitted dose of the recovered dose is greater than 80%. In some embodiments, the emitted dose of the recovered dose is greater than 90%.

In some embodiments, the pharmaceutical composition has a relative standard deviation (RSD) of the homogeneity of the pharmaceutical composition is less than 8%. In some embodiments, the relative standard deviation of the homogeneity of less than 6%. In some embodiments, the relative standard deviation of the homogeneity of less than 4%. In some embodiments, the relative standard deviation of the homogeneity of the pharmaceutical composition is 50% less than the relative standard deviation of the homogeneity of a pharmaceutical composition prepared using other means. In some embodiments, the relative standard deviation of the homogeneity of the pharmaceutical composition is 100% less. In some embodiments, the relative standard deviation of the homogeneity of the pharmaceutical composition is 150% less. In some embodiments, the relative standard deviation of the homogeneity of the pharmaceutical composition is 200% less. In some embodiments, the pharmaceutical composition has a homogeneity from about 95% to about 105%. In some embodiments, the homogeneity is from about 97% to about 103%. In some embodiments, the homogeneity is from about 98% to about 102%. In some embodiments, the relative standard deviation (RSD) of the homogeneity of the pharmaceutical composition is less than 5%. In some embodiments, the relative standard deviation (RSD) of the homogeneity is less than 3%. In some embodiments, the relative standard deviation (RSD) of the homogeneity is less than 1%.

In some embodiments, the pharmaceutical composition has a critical primary pressure that is greater than 10% of an identical pharmaceutical composition prepared by jet milling. In some embodiments, the critical primary pressure is greater than 25%. In some embodiments, the critical primary pressure is greater than 50%.

In some embodiments, the carrier has a Carr's Index of less than 25%. In some embodiments, the Carr's index is less than 20%. In some embodiments, the Carr's index is less than 15%. In some embodiments, the carrier has a tapped density of greater than 250 g/L. In some embodiments, the tapped density is greater than 400 g/L. In some embodiments, the tapped density is greater than 500 g/L. In some embodiments, the carrier has a tapped density from about 250 g/L to about 1500 g/L. In some embodiments, the tapped density is from about 400 g/L to about 1250 g/L. In some embodiments, the tapped density is from about 500 g/L to about 1000 g/L. In some embodiments, the carrier has a poured density of greater than 100 g/L. In some embodiments, the poured density is greater than 150 g/L. In some embodiments, the poured density is greater than 250 g/L. In some embodiments, the carrier has a poured density from about 100 g/L to about 1500 g/L. In some embodiments, the poured density is from about 200 g/L to about 1250 g/L. In some embodiments, the poured density is from about 250 g/L to about 1000 g/L.

In still another aspect, the present disclosure provides pharmaceutical compositions comprising:
(A) an active pharmaceutical ingredient, wherein the active pharmaceutical ingredient is voriconazole, niclosamide, or tacrolimus; and
(B) a carrier, wherein the carrier is a lactose;
wherein the pharmaceutical composition contains one or more particles wherein the active pharmaceutical ingredient has been deposited on the surface of the carrier, the pharmaceutical compositions comprises both the active pharmaceutical ingredient and the carrier in a single particle, and the pharmaceutical composition has a specific surface area that is 50% greater than the specific surface area of the carrier.

In another aspect, the present disclosure provides pharmaceutical compositions comprising:
(A) an active pharmaceutical ingredient, wherein the active pharmaceutical ingredient is antifungal agent, antihelminthic agent, or immunomodulating compound; and
(B) a carrier, wherein the carrier is a sugar;
wherein the pharmaceutical composition contains one or more particles wherein the active pharmaceutical ingredient has been deposited on the surface of the carrier, the pharmaceutical compositions comprises both the active pharmaceutical ingredient and the carrier in a single particle, and the pharmaceutical composition has a specific surface area that is 50% greater than the specific surface area of the carrier.

In another aspect, the present disclosure provides methods of treating a disease or disorder comprising administering to the patient in need thereof a therapeutically effective amount of the pharmaceutical composition described herein, wherein the active pharmaceutical ingredient is useful to treating the disease or disorder.

In still another aspect, the present disclosure provides methods of preventing a disease or disorder comprising administering to the patient in need thereof a therapeutically effective amount of the pharmaceutical composition described herein wherein the active pharmaceutical ingredient is useful to prevent the disease or disorder.

In still yet another aspect, the present disclosure provides kits comprising:
(A) a pharmaceutical composition described herein;
(B) a capsule comprising a unit dose of the pharmaceutical composition, a blister pack comprising a unit dose of the pharmaceutical composition, or a metering device that distributes a unit dose of the pharmaceutical composition; and
(C) an aerosolizing device that disperses the unit dose.

In some embodiments, the aerosolizing device is an inhaler. In some embodiments, the kits comprise capsule comprising a unit dose of the pharmaceutical composition. In other embodiments, the kits comprise a blister pack comprising a unit dose of the pharmaceutical composition. In other embodiments, the kits comprise a metering device that distributes a unit dose of the pharmaceutical composition.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows methods of preparation of the dry powder using suspensions-based TFF. In Method 1, the particles of the carrier are suspended in the drug solution. In Method 2, the particles of the carrier are suspended in the drug-PVP K25 solution. In Method 3, both the particles of the carrier and the engineered particles are suspended in the drug solution.

FIG. 2 shows morphology of inhalation-grade LAC before and after TFF. The x-axis shows that different grades of LAC varied by carrier sizes. Lactohale® LH300 and Lactohale® LH230 showed agglomerated particles, while Respitose® SV003 and Lactohale® LH206 exhibited discrete coarse particles with fine particles on their surface.

FIGS. 3A-3C show morphology of TAC/LAC powders prepared using the suspension based TFF process. (FIG. 3A) TAC/Lactohale® LH230 varied by drug loading. (FIG. 3B) TAC/LAC (10/90) varied by carrier size. (FIG. 3C) TAC/LAC (10/90) with the addition of a secondary excipient. The solid arrows show the location of the LAC. Dot arrows represent some examples of the brittle matrix.

FIG. 4 shows XRD diffractograms of TAC/LAC powders made using the suspension based TFF process.

FIG. 5 shows specific surface area of LAC unprocessed powders (solid dark grey bars), neat LAC powders made using the suspension based TFF process (solid light grey bars), TAC/LAC powders made using the suspension based TFF process (striped bars), and TAC/LAC powder made using conventional blending (dotted bars).

FIGS. 6A & 6B show aerodynamic properties of TAC/Lactohale® LH230 made using the suspension based TFF process versus conventional blending. The x-axis shows drug loading. The y-axis shows (FIG. 6A) MMAD and GSD and (FIG. 6B) FPF and EF (of the recovered dose).

FIGS. 7A & 7B show aerodynamic properties of TAC/Lactohale (10:90) made using the suspension based TFF process versus conventional blending. The x-axis shows the size of the LAC carrier. The y-axis shows (FIG. 7A) MMAD and GSD and (FIG. 7B) FPF and EF (of the recovered dose).

FIG. 7C shows the locations of the recovered drug and the percentage of the drug load that reach different penetration within the respiratory system.

FIGS. 8A & 8B show aerodynamic properties of TAC/Lactohale® LH230 (10/90) with the addition of a secondary excipient made using the suspension based TFF process. (FIG. 8A) MMAD and GSD. (FIG. 8B) FPF and EF (of the recovered dose).

FIG. 9 shows critical primary pressure (CPP) of powders. The five leftmost bars show the CPP of neat material powders made using the suspension based TFF process. The central seven bars show the CPP of TAC-LAC powders made using the suspension based TFF process. The seven rightmost bars show the CPP of TAC-LAC powders made using conventional blending.

FIGS. 10A-10C show morphology of VCZ/LAC powders prepared using the suspension based TFF process. (FIG. 10A) VCZ/Lactohale® LH230 varied by drug loading. (FIG. 10B) VCZ/LAC (10/90) varied by carrier size. (FIG. 10C) VCZ/Lactohale® LH230 (10/90) with the addition of a secondary excipient.

FIG. 11 show XRD diffractograms of VCZ/LAC powders made using the TFF suspension based TFF process.

FIG. 12 shows specific surface area of LAC unprocessed powders (solid dark grey bar), neat LAC powders made using the suspension based TFF process (solid light grey bar), VCZ/LAC powders made using the suspension based TFF process (striped bar), and VCZ/LAC powders made using conventional blending (dotted bar).

FIGS. 13A & 13B show aerodynamic properties of VCZ/Lactohale® LH230 made using the suspension based TFF process versus conventional blending. The x-axis indicates drug loading. (FIG. 13A) MMAD and GSD. (FIG. 13B) FPF and EF (of the recovered dose).

FIGS. 14A & 14B show aerodynamic properties of VCZ/Lactohale (30/70) made using the suspension based TFF process versus conventional blending. The x-axis shows the size of LAC carrier. (FIG. 14A) MMAD and GSD. (FIG. 14B) FPF and EF (of the recovered dose).

FIGS. 15A & 15B show aerodynamic properties of VCZ/Lactohale® LH230 (30/70) with the addition of a secondary excipient made using the suspension based TFF process. (FIG. 15A) MMAD and GSD. (FIG. 15B) FPF and EF (of the recovered dose).

FIG. 16 shows critical primary pressure (CPP) of powders. The five leftmost bars show the CPP of neat material powders made using the suspension based TFF process. The central seven bars show the CPP of VCZ-LAC powders made using the suspension based TFF process. The seven rightmost bars show the CPP of VCZ-LAC powders made using conventional blending.

FIG. 17 shows the particle size of the particles and distribution within the respiratory system for composition before and after storage at ambient conditions for 10 months.

FIG. 18 shows powder x-ray diffraction of those compositions before and after storage at ambient conditions for 10 months.

FIG. 19 shows the particle size of the particles and distribution within the respiratory system for composition with a 1.67% w/w tacrolimus drug loading based upon lactose grade.

FIG. 20 shows the particle size of the particles and distribution within the respiratory system for composition with a 1.67% w/w tacrolimus drug loading with various different solvent systems.

FIG. 21 shows the particle size of the particles and distribution within the respiratory system for composition with a 6.67% w/w tacrolimus drug loading based upon lactose grade.

FIG. 22 shows the particle size of the particles and distribution within the respiratory system for composition with a 6.67% w/w tacrolimus drug loading with various solvent systems.

FIG. 23 shows the particle size of the particles and distribution within the respiratory system for a niclosamide composition.

### DESCRIPTION OF ILLUSTRATIVE EMBODIMENTS

In some aspects, the present disclosure relates to methods of preparing pharmaceutical compositions comprising composite particles containing an active pharmaceutical ingredient and a carrier capable of being delivered to the upper and lower airways in the treatment of diseases. The composite particles are engineered in such a way that the resulting composition may be delivered in powder form using a dry powder inhaler (DPI) to the lower airways. The ability to deliver the pharmaceutical compositions using a range of delivery systems without the need for changes to the powder components and ratios or processing methods makes the composition broadly applicable to a range of patient populations and includes those who are ambulatory or in an out-patient setting, patients with reduced lung function or those who may require mechanical ventilation, and pediatric or geriatric who may exhibit reduced inspiratory capacity. Also provided herein are compositions prepared using these methods. Details of these methods are provided in more detail below.

### I. PHARMACEUTICAL COMPOSITIONS

In some aspects, the present disclosure provides pharmaceutical compositions containing one or more particles wherein an active pharmaceutical ingredient has been deposited on the surface of the carrier and the pharmaceutical compositions comprise both the active pharmaceutical ingredient and the carriers as single particles. Additionally, these particles may be mixed with one or more additional excipients after the initial processing of the active pharmaceutical ingredient and the carrier. These pharmaceutical compositions may further comprise a pharmaceutical composition has been prepared in such a way that the particles may be agglomerated together. In another embodiment, the pharmaceutical compositions may further comprise a pharmaceutical composition has been prepared in such a way that the active pharmaceutical ingredient is present as a discrete domain on the carrier particles. These discrete domains may represent a nanostructured aggregate or other higher order structure to the pharmaceutical composition.

In some embodiments, the pharmaceutical composition may be defined by one or more favorable properties such as the specific surface area, mass median aerodynamic diameter (MMAD), the geometric standard deviation (GSD), fine particle fraction, emitted dose, homogeneity, critical primary pressure, Can's Index, tapped density, or poured density.

The present pharmaceutical compositions prepared according to the methods described herein may have a specific surface area from about 2 m²/g to about 100 m²/g, from about 2.5 m²/g to about 50 m²/g, from about 2.5 m²/g to about 25 m²/g, or from about 2.5 m²/g to about 10 m²/g. The specific surface area of the composition may be from about 2 m²/g, 2.5 m²/g, 3 m²/g, 4 m²/g, 5 m²/g, 6 m²/g, 8 m²/g, 10 m²/g, 12.5 m²/g, 15 m²/g, 20 m²/g, 25 m²/g, 30 m²/g, 40 m²/g, 50 m²/g, 75 m²/g, to about 100 m²/g, or any range derivable therein. The specific surface area may be determined by the single-point Braummer-Emmett-Teller (BET) method using a Monosorb rapid surface area analyzer. Furthermore, the specific surface area of the pharmaceutical compositions prepared using the methods described herein compared to a composition with the same components prepared using conventional powder blending may be 50% greater, 55% greater, 60% greater, 65% greater, 70% greater, 75% greater, 80% greater, 85% greater, 90% greater, 95% greater, 100% greater, or 125% greater.

Similarly, the present pharmaceutical compositions may have a MMAD that is from about from about 1.0 μm to about 10.0 μm, from about 1.5 μm to about 8.0 μm, or from about 2.0 μm to about 6.0 μm. The MMAD may be from about 0.5 μm, 1.0 μm, 1.5 μm, 2.0 μm, 2.5 μm, 3.0 μm, 3.5 μm, 4.0 μm, 4.5 μm, 5.0 μm, 6.0 μm, 7.5 μm, 8.0 μm, to about 10.0 μm, or any range derivable therein. The MMAD may be measured using laser diffraction as described in the Examples below. The MMAD of the pharmaceutical compositions prepared using the methods described herein compared to a composition with the same components prepared using conventional blending may be 20% less, 25% less, 30% less, 35% less, 40% less, 45% less, 50% less, 55% less, 60% less, 65% less, 70% less, 75% less, 80% less, 85% less, 90% less, 95% less, 100% less, or 125% less.

Additionally, the present pharmaceutical compositions may have a GSD that is from about 1.0 to about 10.0, from about 1.25 to about 8.0, or from about 1.5 to about 6.0. The GSD may be from about 0.5, 1.0, 1.5, 2.0, 2.5, 3.0, 3.5, 4.0, 4.5, 5.0, 6.0, 7.5, 8.0, to about 10.0, or any range derivable therein. The GSD may be measured using laser diffraction as described in the Examples below.

Similarly, the pharmaceutical composition may have fine powder fraction of the recovered dose that is greater than the fine powder fraction of a composition that is prepared using other means such as conventional powder blending. The present pharmaceutical compositions prepared using the methods described herein may have a fine powder fraction that is 5% greater, 10% greater, 15% greater, 20% greater, 25% greater, 30% greater, 35% greater, 40% greater, 45% greater, 50% greater, 55% greater, 60% greater, 65% greater, 70% greater, 75% greater, 80% greater, or 90% greater. The fine particle fraction (FPF) of the recovered dose may be calculated as the total amount of drug collected with an aerodynamic diameter below 5 μm as a percentage of the total amount of drug collected. Similarly, the instant composition may have an emitted dose that is greater than 50%, greater than 60%, greater than 70%, greater than 80%, greater than 90%, greater than 95%, greater than 97%, or greater than 98%. The emitted fraction (EF) may be calculated as the total amount of drug emitted from the device as a percentage of total amount of drug collected.

Furthermore, the present compositions preferably have high degree of homogeneity compared to compositions prepared using other methods such conventional powder blending. The present compositions may have a homogeneity from about 95% to about 105%, from about 97% to about 103%, or from about 98% to about 102%. The homogeneity may be from about 90%, 92%, 95%, 96%, 97%, 98%, 99%, 100%, 101%, 102%, 103% 104%, 105%, 108%, or to about 110%, or any range derivable therein. Furthermore, the relative standard deviation of the homogeneity is less than 10%, less than 8%, less than 6%, less than 5%, less than 4%, less than 3%, less than 2%, or less than 1%. The homogeneity may be determined by performing the assay of drug in bulk powder and reported as the percentage of drug to the nominal dose. The relative standard deviation of the homogeneity may be calculated by the standard deviation of the drug percentage divided by the average of drug percentage. In some embodiments, the relative standard deviation of the homogeneity of the pharmaceutical compositions prepared using the present methods is less those prepared using conventional methods. The relative standard deviation of the homogeneity may be about 25% less, 30% less, 40% less, 50% less, 60% less, 75% less, 80% less, 100% less, 120% less, 125% less, 140% less, 150% less, 160% less, 175% less, 180% less, 200% less, or about 250% less.

Furthermore, the pharmaceutical compositions when formulated into an inhaler or other similar device may have a critical primary pressure that is greater than a similar composition prepared by jet milling. The critical primary pressure represents a pressure that overcomes interparticulate forces and disperses powder to primary particles or smaller agglomerates. The critical primary pressure may be 5% greater, 10% greater, 15% greater, 20% greater, 25% greater, 30% greater, 40% greater, 50% greater, or 75% greater.

Finally, the present pharmaceutical compositions may have a Carr's Index that is less than 30%, less than 25%, less than 20%, or less than 15%. Similarly, the composition may have a tapped density that is greater than 200 g/L, greater than 250 g/L, greater than 300 g/L, greater than 350 g/L, greater than 400 g/L, greater than 450 g/L, greater than 500 g/L, or greater than 750 g/L. The tapped density may be from about 250 g/L to about 1500 g/L, from about 400 g/L to about 1250 g/L, or from about 500 g/L to about 1000 g/L. The tapped density may be from about 200 g/L, 250 g/L, 300 g/L, 400 g/L, 450 g/L, 500 g/L, 550 g/L, 600 g/L, 700 g/L, 750 g/L, 800 g/L, 900 g/L, 1,000 g/L, 1,250 g/L, 1,400 g/L, 1,500 g/L, to about 1,600 g/L, or any range derivable therein. The poured density of the pharmaceutical composition may be from about 100 g/L to about 1500 g/L, from about 200 g/L to about 1250 g/L, or from about 250 g/L to about 1000 g/L. The poured density of the pharmaceutical composition may be from about 50 g/L, 100 g/L, 150 g/L, 200 g/L, 250 g/L, 300 g/L, 400 g/L, 450 g/L, 500 g/L, 550 g/L, 600 g/L, 700 g/L, 750 g/L, 800 g/L, 900 g/L, 1,000 g/L, 1,250 g/L, 1,400 g/L, 1,500 g/L, to about 1,600 g/L, or any range derivable therein. The poured density may be greater than about 100 g/L, 150 g/L, 200 g/L, 250 g/L, or 300 g/L. The poured and tapped density are measured according to a method modified from USP <616> method using a Tapped Density Tester and a 10-mL graduated cylinder. Carr's (Compressibility) index are calculated based on USP General Chapter <616>.

A. Active Pharmaceutical Ingredient

The “active pharmaceutical ingredient” used in the present methods refers to any substance, compound, drug, medicament, or other primary active ingredient that provides a therapeutic or pharmacological effect when administered to a human or animal. In some embodiments, the pharmaceutical composition comprises from about 1% w/w to about 50% w/w, from about 2.5% w/w to about 40% w/w, from about 5% w/w to about 35% w/w, or from about 0.5% w/w, 1% w/w, 1.5% w/w, 2% w/w, 2.5% w/w, 5% w/w, 10% w/w, 15% w/w, 20% w/w, 30% w/w, 40% w/w, to about 50% w/w of the active pharmaceutical ingredient, or any range derivable therein. In some embodiments, at least 60%, 80%, 85%, 90%, 95%, 97%, 98%, or 99% of the active pharmaceutical ingredient is in amorphous form. In other embodiments, at least 60%, 80%, 85%, 90%, 95%, 97%, 98%, or 99% of the active pharmaceutical ingredient is in crystalline form.

Suitable active pharmaceutical ingredients may be any biologically active agents or a salt, isomer, ester, ether or other derivative, including prodrug, thereof, which include, but are not limited to, anticancer agents, antifungal agents, psychiatric agents such as analgesics, consciousness level-altering agents such as anesthetic agents or hypnotics, nonsteroidal anti-inflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta agonists, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the active pharmaceutical ingredients may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxanide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzo ate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the active pharmaceutical ingredients may be voriconazole or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, posaconazole, voriconazole, terconazole and c) thiazoles such as abafungin. Other drugs that may be used with this approach include, but are not limited to, hyperthyroid drugs such as carimazole, anticancer agents like cytotoxic agents such as epipodophyllotoxin derivatives, taxanes, bleomycin, anthracyclines, as well as platinum compounds and camptothecin analogs. The following active pharmaceutical ingredients may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The agents may also include a psychiatric agent such as an antipsychotic, anti-depressive agent, or analgesic and/or tranquilizing agents such as benzodiazepines. The agents may also include a consciousness level-altering agent or an anesthetic agent, such as propofol. The present compositions and the methods of making them may be used to prepare pharmaceutical compositions with the appropriate pharmacokinetic properties for use as therapeutics.

In some aspects, the pharmaceutically active ingredient is an immune system modulating compound. The compound may be an immunosuppressive agent such as tacrolimus. Tacrolimus (TAC) is a widely used immunosuppressive agent isolated from Streptomyces tsukubaensis. It has proven to be a potent immunosuppressant in transplantation medicine for treatment of organ rejection and different immunological diseases such as pulmonary fibrosis and bronchiolar asthma. TAC was first introduced as rescue therapy when cyclosporin A (CsA) therapy failed to prevent graft rejection. It has a mechanism of action similar to that of CsA, but its immunosuppressive activity is 10- to 100-times more potent than CsA. TAC is currently available in both an intravenous and oral dosage form (commercially known as Prograf®). However, these current available dosage forms of the drug are poorly tolerated and provide a variable and/or low bioavailability. The oral formulations of TAC present a considerable challenge as the drugs are practically insoluble in water and extensively metabolized from both CYP3A4 metabolism and p-glycoprotein efflux transport within the intestinal epithelium. The oral bioavailability of TAC varies from 4% to 93%. Inefficient or erratic drug absorption is primarily the result of incomplete absorption from the gastrointestinal tract and first-pass metabolism, which is subject to considerable inter-individual variation.

In some embodiments, the active pharmaceutical ingredient is niclosamide. Niclosamide is a poorly water soluble, lipophilic molecule previously known to have poor and variable bioavailability which for its current approved indication for treating helminthic infections in the gastrointestinal tract is not a limiting factor. When attempting to repurpose the medication for the treatment of diseases such as prostate cancer or viral infections, which require systemic concentrations and/or lung concentrations of the drug, the challenges to overcome the bioavailability limitations become clear. As niclosamide is both poorly water soluble and lipophilic, the rate limiting step for the oral absorption of the drug is the dissolution of the molecule. This drug also has a number of other potential uses including as a treatment of viral infections such as SARS-CoV-2 and MERS.

Unfortunately, the majority of drugs that show pharmacological activity against cancers in vitro are poorly water-soluble and thus exhibit poor or no bioavailability. While often not a limitation for their currently approved indications, their usefulness in treating cancers often requires significantly better absorption of the drugs to achieve drug concentrations sufficient for tumor inhibition. These pharmaceutical compositions need mechanisms that may be used to overcome the limitations of solubility by the pharmaceutical industry in 19 commercial products approved by the Food & Drug Administration between 2007 and 2017.

B. Inhalation

In some embodiments, the present disclosure relates to respirable particles must be within a particular aerodynamic size range. In some embodiments, the pharmaceutical composition has a MMAD of from about 1.0 to 10.0 microns, from about or 1.5 to about 8 microns, from about 2.0 to about 6.0 microns, or from about 0.5 microns, 1.0 microns, 1.5 microns, 2.0 microns, 2.5 microns, 3.0 microns, 3.5 microns, 4.0 microns, 4.5 microns, 5.0 microns, 6.0 microns, 8.0 microns, 10.0 microns, to about 15.0 microns, or any range derivable therein. In some embodiments, the present disclosure provides methods for the administration of the inhalable pharmaceutical composition provided herein using a device. Administration may be, but is not limited, to inhalation of pharmaceutical using an inhaler. In some embodiments, an inhaler is a simple passive dry powder inhaler (DPI), such as a Plastiape RS01 monodose DPI. In a conventional dry powder inhaler, dry powder is stored in a capsule or reservoir and is delivered to the lungs by inhalation without the use of propellants.

In some embodiments, an inhaler is a single use, disposable inhaler such as a single-dose DPI, such as a DoseOne™, Spinhaler, Rotohaler®, Aerolizer®, or Handihaler. These dry powder inhalers may be a passive DPI. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination. In some embodiments, the inhalable pharmaceutical composition is delivered as a propellant formulation, such as HFA propellants.

In some embodiments, an inhaler is a nebulizer or a soft-mist inhaler such as those described in PCT Publication No. WO 1991/14468 and WO 1997/12687, which are incorporated herein by reference. A nebulizer is used to deliver medication in the form of an aerosolized mist inhaled into the lungs. The medication formulation be aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. In some embodiments, the single use, disposable nebulizer may be used herein. A nebulizer may utilize a flow rate of between about 3-12 L/min, such as about 6 L/min. In some embodiments, the nebulizer is a dry powder nebulizer.

In some embodiments, the composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration four times a day, three times a day, twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, the pharmaceutical composition is administered once per day. In preferred embodiments, the pharmaceutical composition is administered less than once per day, such as every other day, every third day, or once per week. I

In some embodiments, the amount of the pharmaceutical composition of the nebulizer or inhaler may be provided in a unit dosage form, such as in a capsule, blister or a cartridge, wherein the unit dose comprises at least 0.05 mg of the pharmaceutical composition, such as at least 0.075 mg or 0.100 mg of the pharmaceutical composition per dose. In particular aspects, the unit dosage form does not comprise the administration or addition of any excipient and is merely used to hold the powder for inhalation (i.e., the capsule, blister, or cartridge is not administered). In some embodiments, the entire amount of the powder load may be administered in a high emitted dose, such as at least 1 mg, preferably at least 10 mg, even more preferably 50 mg. In some embodiments, administration of the powder load results in a high fine particle dose into the deep lung such as greater than 1 mg. Preferably, the fine particle dose into the deep lung is at least 5 mg, even more preferably at least 10 mg. In some embodiments, the dose may further comprise using a dose from a reservoir or non-unit dose form and the relevant dose is metered out from the device such as a Turbuhaler.

C. Excipients & Carriers

In some aspects, the present disclosure comprises one or more excipients formulated into pharmaceutical compositions. An “excipient,” also commonly known as pharmaceutically acceptable carriers, diluents or bulking agents, are relatively inert substances used to facilitate administration or delivery of an API into a subject or used to facilitate processing of an API into drug formulations that can be used pharmaceutically for delivery to the site of action in a subject. Furthermore, these compounds may be used as diluents in order to obtain a dosage that can be readily measured or administered to a patient. Non-limiting examples of excipients include polymers, stabilizing agents, surfactants, surface modifiers, solubility enhancers, buffers, encapsulating agents, antioxidants, preservatives, nonionic, anionic and cationic wetting or clarifying agents, viscosity increasing agents, pH adjusting agents and absorption-enhancing agents. In some embodiments, the pharmaceutical composition comprises from about 50% w/w to about 99% w/w, from about 60% w/w to about 95% w/w, from about 65% w/w to about 90% w/w, or from about 40% w/w, 45% w/w, 50% w/w, 55% w/w, 60% w/w, 65% w/w, 70% w/w, 75% w/w, 80% w/w, 85% w/w, 80% w/w, 92% w/w, 94% w/w, 95% w/w, 97% w/w, to about 99% w/w of the carrier, or any range derivable therein. In some embodiments, at least 60%, 80%, 85%, 90%, 95%, 97%, 98%, or 99% of the carrier is in amorphous form. In other embodiments, at least 60%, 80%, 85%, 90%, 95%, 97%, 98%, or 99% of the carrier is in crystalline form.

In some aspects, the pharmaceutical compositions of the present disclosure may further comprise one or more carriers, such as a sugar or sugar alcohol. The compositions may also further comprise one or more additional excipients such as a lubricant, a glidant, or an amino acid. Furthermore, one or more flow enhancing agents such as magnesium salts may be used. A non-limiting example of a flow enhancing agent is magnesium stearate. In other embodiments, the compositions may further comprise one or more silicon dioxides or silicas. Such silica could be a fumed silica or another form of silica that is approved for use in inhalation treatments. In other aspects, larger molecules like amino acids, peptides and proteins are incorporated to facilitate inhalation delivery, including leucine, trileucine, histidine and others. Some non-limiting examples of amino acids include hydrophobic amino acids, such as leucine.

Some compositions may further comprise a mixture of two or more excipients. In some embodiments, the amount of the further excipient may be from about 0.05% w/w to about 50% w/w, from about 1% w/w to about 15% w/w, or from about 2.5% w/w to about 10% w/w. In some embodiments, the amount of the additional excipient is from about 0.05% w/w, 0.1% w/w, 0.25% w/w, 0.5% w/w, 0.75% w/w, 1.0% w/w, 1.5% w/w, 2.0% w/w, 2.5% w/w, 3.0% w/w, 4.0% w/w, 5.0% w/w, 6.0% w/w, 8.0% w/w, 10% w/w, 15% w/w, 20% w/w, 25% w/w, 30% w/w, 40% w/w, to about 50% w/w, or any range derivable therein.

1. Saccharide Carriers

In some aspects, the present disclosure comprises one or more excipients as carriers formulated into pharmaceutical compositions. These excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In some aspects, the carriers used herein are at least sparingly soluble in the solvent used to prepare the pharmaceutical composition. The carriers may slightly soluble, very slightly soluble, or practically insoluble. The solubility of the carrier in the solvent system are described using the solubility standards established in the U.S. Pharmacopeia.

2. Polymers

In some embodiments, the excipient is a pharmaceutically acceptable polymer. In some embodiments, the excipient is a non-cellulosic polymer. In some embodiments, the excipient is a non-ionizable non cellulosic polymer, such as polyvinylpyrrolidone. In some embodiments, the polyvinylpyrrolidone has a molecular weight from about 10,000 to about 40,000 or from about 20,000 to about 30,000. In some embodiments, the polyvinylpyrrolidone has a molecular weight from about 10,000, 12,000, 14,000, 16,000, 18,000, 20,000, 22,000, 24,000, 26,000, 28,000, 30,000, 32,000, 34,000, 36,000, 38,000, to about 40,000, or any range derivable therein. In some embodiments the polyvinylpyrrolidone has a molecular weight of about 24,000.

### II. MANUFACTURING METHODS

A. Thin-Film Freezing

Without wishing to be bound by any theory, it is believed that this process may be used to introduce the particles into a single particle containing one or more active pharmaceutical ingredients and the carrier into the same particle. In particular, if multiple therapeutic agents are present in the composition, the particles contain two or more of the active pharmaceutical ingredients. The particles obtained from this process may exhibit one or more beneficial properties for administration via inhalation such as a high surface area, a low tapped density, a low poured density, or improved flowability or compressibility such as a low Carr's Index. The method comprises dissolving the active pharmaceutical ingredients into a solvent. The solvent may be an organic solvent such as acetonitrile, dioxane, or an alcohol such as isopropanol or butanol. The organic solvent is a polar aprotic solvent wherein the solvent lacks an acidic proton but contains one or more polar bonds. These solvents may also include tetrahydrofuran, dimethylformamide, or dimethylsulfoxide. In some embodiments, the solvent may be a mixture of two or more solvents.

In some embodiments, the method further comprises using a surface that has been cooled to a first reduced temperature. In some embodiments, the first reduced temperature is from about 25° C. to about −120° C., from about −20° C. to about −100° C., from about −60° C. to about −90° C., or from about −150° C., −125° C., −120° C., −110° C., −100° C., −75° C., −50° C., −25° C., 0° C., to about 25° C., or any range derivable therein. In some embodiments, the pharmaceutical mixture is applied from a height from about 1 cm to about 250 cm, from about 2.5 cm to about 100 cm, from about 5 cm to about 50 cm, or from about 0.5 cm, 1 cm, 1.5 cm, 2 cm, 2.5 cm, 5 cm, 10 cm, 15 cm, 20 cm, 25 cm, 50 cm, 75 cm, 100 cm, 150 cm, 200 cm, 250 cm, to about 300 cm, or any range derivable therein. In some embodiments, the surface rotates at a speed. In some embodiments, the speed is from about 5 rpm to about 500 rpm, from about 25 rpm to about 400 rpm, from about 50 rpm to about 250 rpm, from about 50 rpm to about 150 rpm, or from about 5 rpm, 10 rpm, 15 rpm, 20 rpm, 25 rpm, 50 rpm, 75 rpm, 100 rpm, 150 rpm, 200 rpm, 250 rpm, 300 rpm, 400 rpm, to about 500 rpm, or any range derivable therein.

In some embodiments, the drying process comprises lyophilization. In some embodiments, the drying process comprises two drying cycles. In some embodiments, the first drying cycle comprises drying at a first temperature from about −120° C. to about 0° C., from about −10° C. to about −80° C., from about −20° C. to about −60° C., or from about −150° C., −125° C., −120° C., −110° C., −100° C., −90° C., −80° C., −70° C., −60° C., −50° C., −40° C., −30° C., −20° C., −10° C., to about 0° C., or any range derivable therein. In some embodiments, the pharmaceutical composition is dried at a first reduced pressure from about 10 mTorr to 500 mTorr, from about 25 mTorr to about 250 mTorr, from about 50 mTorr to about 150 mTorr, or from about 5 mTorr, 6 mTorr, 7 mTorr, 8 mTorr, 9 mTorr, 10 mTorr, 20 mTorr, 25 mTorr, 50 mTorr, 100 mTorr, 150 mTorr, 200 mTorr, 250 mTorr, 300 mTorr, 350 mTorr, 400 mTorr, 450 mTorr, to about 500 mTorr, or any range derivable therein.

In some embodiments, the second drying cycle comprises drying at a second temperature from about 0° C. to about 80° C., from about 10° C. to about 60° C., from about 20° C. to about 50° C., or from about 0° C., 10° C., 20° C., 30° C., 40° C., 50° C., 60° C., 70° C., to about 80° C., or any range derivable the rein. In some embodiments, the second drying cycle comprises drying at a reduced pressure. In some embodiments, the pharmaceutical composition is dried at a second reduced pressure from about 10 mTorr to 500 mTorr, from about 25 mTorr to about 250 mTorr, from about 50 mTorr to about 150 mTorr, or from about 10 mTorr, 15 mTorr, 20 mTorr, 25 mTorr, 50 mTorr, 75 mTorr, 100 mTorr, 150 mTorr, 200 mTorr, 250 mTorr, 300 mTorr, 350 mTorr, 400 mTorr, 450 mTorr, to about 500 mTorr, or any range derivable therein.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, “therapeutically active agent”, or “pharmaceutical active ingredient” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition. A “dispersion” is a solution in which one or more of the compounds is not dissolved in the solution but rather is only soluble or less. In particular, the compound may only be sparingly soluble, slightly soluble, or very slightly soluble.

The term “solubility” is defined the amount of a compound that can be dissolved in a solvent. In particular, the particular amount may be described using the U.S. Pharmacopeia descriptive terms. In particular, the term “very soluble” means that less than 1 part of solvent is required for 1 part of solute. The term “freely soluble” means from 1 to 10 parts of solvent is required for 1 part of solute. The term “soluble” means from 10 to 30 parts of solvent is required for 1 part of solute. The term “sparingly soluble” means from 30 to 100 parts of solvent is required for 1 part of solute. The term “slightly soluble” means from 100 to 1000 parts of solvent is required for 1 part of solute. The term “very slightly soluble” means from 1000 to 10,000 parts of solvent is required for 1 part of solute. The term “practically insoluble or insoluble” means more than 10,000 parts of solvent is required for 1 part of solute.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pg. 2).

As used herein, the term “physiological pH” refers to a solution with is at its normal pH in the average human. In most situations, the solution has a pH of approximately 7.4.

As used herein, “inhalation” or “pulmonary inhalation” is used to refer to administration of pharmaceutical preparations by inhalation so that they reach the lungs and in particular embodiments the alveolar regions of the lung. Typically, inhalation is through the mouth, but in alternative embodiments in can entail inhalation through the nose.

As used herein, “dry powder” refers to a fine particulate composition that is not suspended or dissolved in an aqueous liquid.

A “non-complex dry powder inhaler” refers to a device for the delivery of medication to the respiratory tract, in which the medication is delivered as a dry powder in a single-use, single-dose manner. In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter) and is measured by cascade impaction, such as by a Next Generation Impactor (NGI apparatus).

The term “amorphous” refers to a substantially noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Ordered Mixing by Suspension based Thin Film Freezing to Prepare Dry Powders for Inhalation

A. Experimental Design

The development of inhaled products must address several physical difficulties to achieve effective drug delivery. The aerodynamic diameter of drug particles must be between 1 μm and 5 μm to maximize the probability of drug particles from a DPI reaching the lower respiratory tract (Prime et al., 1997). However, such micronized drug particles have high forces of cohesiveness and a tendency to agglomerate, which results in poor flowability, poor aerosolization properties, and high dose variability (Chan and Chew, 2003).

To overcome problems associated with the development of inhaled products, such as aerodynamic diameter, flowability, aerosolization properties, and dose variability, the ordered mixture concept has been applied to prepare carrier-based formulations for pulmonary drug delivery. The carrier-based formulation is composed of micronized drug particles adhered to a coarse carrier such as lactose (LAC). In this system, drug particles are deaggregated from the carrier particles during aerosolization, which introduces highly cohesive micronized drug particles to the deep lung (de Boer et al., 2012). Carriers can enhance drug particle flowability, reduce the aggregation of drug particles, and aid in dispersion and aerosolization. This improves dose accuracy and minimizes the dose variability compared to the drug alone. It also makes them easier to handle during the manufacturing process (de Boer et al., 2012). Unlike random mixtures, the effect of gravity is limited in the ordered mixture, thus minimizing the freedom of migration for fine or adhesive particles (Tan et al., 2019). Additionally, the interactions between fine drug particles and the surface of a coarse carrier (i.e., interactions governed by van der Waals, capillary, electrostatic, and mechanical forces) improve the uniformity of drug distribution and the handling of the powder blend (de Boer et al., 2012).

Although the ordered mixture concept was intended to improve powder homogeneity, controlling the interparticulate forces between the micronized drug and the carrier continues to be a challenge for the development of carrier-based mixtures. It has been reported that the blending process should be optimized in order to create the desired blend organization with an optimal cohesive-adhesive balance (Tan et al., 2019; Begat et al., 2004) because blending affects the physical rearrangement and the interparticulate forces between the drug and the carrier, which can subsequently affect the aerosolization of carrier-based DPI formulations (Begat et al., 2004). The adhesion force between the drug and carrier must be strong enough to maintain the blend homogeneity during manufacturing process, but it should not be too strong to make drug particles hard to detach by inhalation flow (Zhou and Morton, 2012). The adhesive tendency of drug particles in an ordered mixture can increase with blending time (Grasmeijer et al., 2013). High drug-carrier adhesive forces can lead to inadequate detachment of the drug from the carrier, thus causing poor drug deposition efficiency in drug-carrier DPI formulations (de Boer et al., 2012).

Blend homogeneity is a critical attribute for an ordered mixture, especially for low-dose formulations and high-potency drugs. Very low doses of API impose strict requirements for content uniformity (Sarkar et al., 2017). The quality of the blend may also be greatly affected by the electrostatic charge on the surface of the particles, which is generated by friction between the particles or between the particles and the blender surface during blending (Kaialy, 2016; Pu et al., 2009). Since fine particles tend to adhere to everything during the blending process (e.g., the blender, container wall, impeller wings), fine particle adhesion leads to loss of the drug (with subsequent inhomogeneity) and segregation tendency (Sarkar et al., 2017). In many cases, inadequate mixing cannot be improved simply by increasing the mixing time. Other factors (e.g., mixer selection, rotation speed, fill level) have been considered to improve blend homogeneity. Furthermore, prolonged blending times may also cause de-mixing, which occurs when the powder is mixed beyond a critical blending time (Poux et al., 1991). Grasmeijer et al. reported that prolonged mixing time decreased the content uniformity of salmeterol and fluticasone (Grasmeijer et al., 2013). De-mixing is related to excessive inertial or shear forces, which lead to the disruption of the adhesive forces between the drug and the carrier, thus increasing the segregation potential (Staniforth et al., 1981).

Many studies have extensively investigated the factors that improve content homogeneity while maintaining detachment of the drug from the carrier. Optimization of carrier particle size is one strategy; however, the effect of carrier size on aerosol performance is complicated and not completely understood. Some studies have reported that a small particle size of the carrier can increase the respirable dose (Kaialy et al., 2012; Le et al., 2012), but others have demonstrated that an increase in carrier size does not always negatively affect drug aerosolization (Kaialy et al., 2013; Hassan and Lau, 2010). Additionally, the use of small carrier size must address the disadvantages of small particles. Smaller carrier size leads to high variation in content uniformity (RSD>8.0%) (Kaialy et al., 2012).

Modification of the surface roughness of a carrier has been proposed as another strategy to change the contact area between the drug and carriers, which affects particle interaction (Zhou and Morton, 2012). The addition of a fine LAC carrier to the larger carrier particles has been reported as one way to modify the active sites (Zeng et al., 1999; Young et al., 2007; Tee et al., 2000; Adi et al., 2008). There was a linear relationship between the fine particle fraction and the content of fine LAC when it was less than 15% (Young et al., 2007). It has been proposed that these fine particles can preferentially adhere to the active sites, thus minimizing the area that drug particles can adhere to (Zeng et al., 2000). Despite an improvement in drug dispersibility, Zeng et al. reported that the addition of fine LAC particles to a mixture of coarse LAC and micronized drug significantly reduced the content uniformity of the drug. Therefore, optimization of mixing time and the order of mixing are required to obtain homogeneous powders (Zeng et al., 2000; Jones et al., 2010).

The addition of force-control agents such as leucine or magnesium stearate is another way to reduce surface passivation of high surface free energy sites, which can subsequently improve DPI performance (Singh et al., 2015; Begat et al., 2005). Nevertheless, the blending homogeneity is influenced by the carrier particle surface roughness (Karner et al., 2014). Karner et al. reported that the content uniformity of the mixture containing smooth carriers was higher than the mixture containing a rough surface of LAC (Karner et al., 2014). It was hypothesized that the drug is less likely to attach to the rougher surface, leading to weaker adhesion forces between the drug and the rough surface after mixing (Karner et al., 2014). Thus, this may minimize the blending homogeneity (Karner et al., 2014).

Although carrier-based mixtures can be successfully developed using the complex optimization of interparticulate forces between the micronized drug and its carrier, the translation of lab-scale formulations into a commercially inhaled product is also not straightforward (Sarkar et al., 2017). The scaling up of a drug-carrier blend requires a robust manufacturing process (Sarkar et al., 2017). Batch size has a strong impact on blend uniformity, hence the manufacturing of different batch sizes must also optimize processing parameters (e.g., mixing times, mixing speed, type of mixing). Moreover, several studies have reported that the variation in a DPI product is mainly attributable to the batch-to-batch consistency of the raw material LAC (de Boer et al., 2012; Steckel et al., 2004). Such batch-to-batch variations in the carrier included the difference in fine particle content, particle size distribution, surface morphology, and amorphous content (Steckel et al., 2004).

Since the majority of currently marketed DPI products exhibited a relatively low pulmonary deposition (˜10-35% fine particle fraction) (Crowder et al., 2002), particle engineering has been applied to improve the aerosol performance of DPI products. TFF is one of the bottom-up particle engineering techniques that can modify the physicochemical properties of the drug, such as particle size, surface characteristics, morphology, and crystallinity (Overhoff et al., 2009). In some cases (e.g., voriconazole), the drug and excipient are formed as nanoaggregates. The excipient (e.g., mannitol) functions as a surface modifier to minimize the cohesivity between the drug particles and subsequently improve drug dispersibility (Moon et al., 2019). In other cases (e.g., tacrolimus), TFF can produce amorphous drugs as a brittle nanostructured matrix, which is an agglomeration of the linkage of the nanoparticles formed from dissolved API (Watts et al., 2013). In this system, a shear force from the device and from inhalation flow can break down the brittle matrix of porous particles into low-density, respirable particles (Watts et al., 2013). TFF particles provide several advantages over micronized drug particles produced by milling. Wang et al. reported that TFF particles, which have a large particle size (>10 μm), can avoid macrophage uptake, thus prolonging the drug retention in the lung (Wang et al., 2014). Moreover, nanoaggregates are more uniformly distributed in the lung than microparticles (Longest et al., 2017).

To avoid these reported homogeneity problems observed in conventional powder blending, we investigated the feasibility of TFF in preparing ordered mixtures for inhalation in one single step using several model drugs: niclosamide (NIC), tacrolimus (TAC) and voriconazole (VCZ). In this system, a drug is dissolved in the solvent, then lactose (LAC) carrier particles are dispersed (i.e., suspended) into the same solvent, which is an antisolvent for LAC. It was hypothesized that the nanostructured brittle matrix or nanoaggregates can strongly agglomerate with or onto a carrier during the TFF process, which can subsequently improve the homogeneity, density, flowability and handling of the powder blend. In the meantime, using formulation optimization, the API in the TFF ordered mixture powder could disperse from a carrier upon aerosolization and exhibit optimal aerosol performance. Furthermore, the effect of carrier size, drug loading, and the presence of secondary excipients on aerosol performance and homogeneity were evaluated.

### B. Materials and Methods

Materials. Tacrolimus USP was purchased from Apotex Fermentation Inc. (Winnipeg, Manitoba, Canada). Voriconazole USP was purchased from Aurobindo Pharma Limited (Telangana, India). Trifluoroacetic acid, phosphoric acid, acetonitrile (HPLC grade), methanol (HPLC grade), and 1,4-dioxane were purchased from Fisher Scientific (Fair Lawn, N.J., USA). Lactohale® (LH300, LH230, and LH206) and Respitose® SV003 were purchased from DFE Pharma (Goch, Germany). Povidone K25 was kindly provided by BASF (Florham Park, N.J., USA). Quali-V®-1 HPMC capsules (size 3) were kindly provided by Qualicaps®, Inc (USA). RS01 and RS00 high-resistance monodose dry powder inhalers were kindly provided by Plastiape S.p.A. (Osnago, Italy).

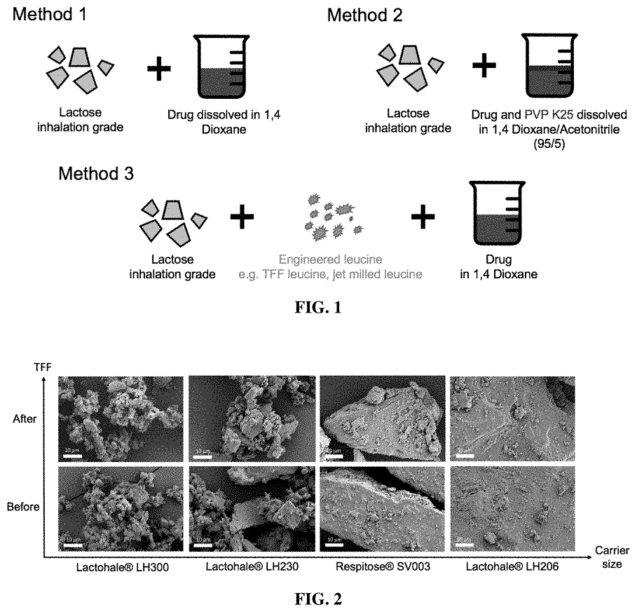

Preparation of powder for dry powder inhalation using suspension based TFF process. The dispersion of TAC or VCZ was prepared using three different methods based on the formulation compositions (FIG. 1). In the first method, a drug was dissolved in 1,4-dioxane. Then, the LAC carrier was dispersed into the solution. In the second method, the drug and was dissolved in 1,4-dioxane. Polyvinylpyrrolidone (PVP) K25 was dissolved in acetonitrile. Then, both solutions were mixed to obtain 1,4-dioxane-acetonitrile (95:5 v/v) before dispersing the LAC carrier into the solution. In the third method, TFF neat leucine was prepared using TFF of the leucine solution (1.0% leucine in water) at −80° C., while jet-milled leucine was prepared as described in Section 2.3. A drug was dissolved in 1,4-dioxane. Then, engineered leucine (TFF leucine or jet-milled leucine) and the LAC carrier were dispersed in the solution.

The grade of the LAC, the drug loading, and the percentage of secondary excipient (including PVP K25, TFF leucine, and jet-milled leucine) was optimized as shown in Table 1. Each dispersion was shaken upon dropping, then dropped from a height of 10 cm onto a rotating cryogenic stainless-steel drum. All samples were frozen at −80±10° C. and then transferred to a lyophilizer. The primary drying cycle was performed at −40° C. and 100 mTorr for 20 h, and the secondary drying cycle was held at 40° C. and 100 mTorr for 20 h.

TABLE 1
Formulation compositions of TAC-LAC powders and VCZ-TAC
powders made using the suspension based TFF process.

| | | % | LAC | | | % TFF | % Milled | |
|---|---|---|---|---|---|---|---|---|
| Formulation | Drug | Drug | grade | % LAC | % PVP | leucine | leucine | Solvent |
| TAC 1 | TAC | 1 | LH230 | 99 | — | — | — | Dioxane |
| TAC 2 | | 5 | LH230 | 95 | — | — | — | |
| TAC 3 | | 10 | LH230 | 90 | — | — | — | |
| TAC 4 | | 20 | LH230 | 80 | — | — | — | |
| TAC 5 | | 30 | LH230 | 70 | — | — | — | |
| TAC 6 | | 10 | LH300 | 90 | — | — | — | |
| TAC 7 | | 10 | SV003 | 90 | — | — | — | |
| TAC 8 | | 10 | LH206 | 90 | — | — | — | |
| TAC 9 | | 10 | LH230 | 90 | — | — | — | Dioxane- |
| TAC 10 | | 10 | LH230 | 90 | 2.5 | — | — | Acetonitrile |
| TAC 11 | | 10 | LH230 | 90 | 5 | — | — | (95:5) |
| TAC 12 | | 10 | LH230 | 90 | — | 5 | — | Dioxane |
| TAC 13 | | 10 | LH230 | 90 | — | 10 | — | |
| TAC 14 | | 10 | LH230 | 90 | — | — | 10 | |
| TAC 15 | | 100 | — | — | — | — | — | Dioxane |
| VCZ 1 | VCZ | 1 | LH230 | 99 | — | — | — | Dioxane |
| VCZ 2 | | 5 | LH230 | 95 | — | — | — | |
| VCZ 3 | | 10 | LH230 | 90 | — | — | — | |
| VCZ 4 | | 20 | LH230 | 80 | — | — | — | |
| VCZ 5 | | 30 | LH230 | 70 | — | — | — | |
| VCZ 6 | | 1 | LH230 | 99 | — | 10 | — | |
| VCZ 7 | | 1 | LH230 | 99 | — | — | 10 | |
| VCZ 8 | | 30 | LH300 | 70 | — | — | — | |
| VCZ 9 | | 30 | SV003 | 70 | — | — | — | |
| VCZ 10 | | 30 | LH206 | 70 | — | — | — | |
| VCZ 11 | | 30 | LH230 | 70 | — | — | — | Dioxane- |
| VCZ 12 | | 30 | LH230 | 70 | 2.5 | — | — | Acetonitrile |
| VCZ 13 | | 30 | LH230 | 70 | 5 | — | — | (95:5) |
| VCZ 14 | | 30 | LH230 | 70 | — | 5 | — | |
| VCZ 15 | | 30 | LH230 | 70 | — | 10 | — | Dioxane |
| VCZ 16 | | 30 | LH230 | 70 | — | — | 10 | |
| VCZ 17 | | 100 | — | — | — | — | — | Dioxane |

Jet-milling of TAC, VCZ, and leucine. TAC, VCZ, and leucine were micronized using a lab-scale Alijet air jet mill (a model 00 Jet-O-Mizer, Fluid Energy, Telford, Pa.) to a particle size distribution within the respirable range of 1-5 μm for TAC and VCZ and a particle size range of 6-10 μm for leucine. The air jet mill was set at 75 psi grind pressure, 65 psi feed pressure, and 0.7 g/min feed rate.

Blending of jet-milled drug with LAC carrier. Powder blends of inhalation-grade LAC and milled TAC or milled VCZ were prepared using a V-shape blender (MaxiBlend® Lab Blender, GlobePharma, New Brunswick, N.J., USA). These powders contained various drug loadings, and the various grades of LAC were prepared as shown in Table 2. The powders were blended at 25 rpm for 5 min.

TABLE 2
Formulation compositions of TAC/LAC powders and
VCZ/LAC powders made using conventional blending.

| | Drug | % Drug | LAC grade | % LAC |
|---|---|---|---|---|
| Milled TAC | Jet-milled TAC | 1 | Lactohale ® LH230 | 99 |
| Blend 1 | | | | |
| Milled TAC | | 10 | Lactohale ® LH230 | 90 |
| Blend 2 | | | | |
| Milled TAC | | 30 | Lactohale ® LH230 | 70 |
| Blend 3 | | | | |
| Milled TAC | | 10 | Lactohale ® LH300 | 90 |
| Blend 4 | | | | |
| Milled TAC | | 10 | Respitose ® SV003 | 90 |
| Blend 5 | | | | |
| Milled TAC | | 10 | Lactohale ® LH206 | 90 |
| Blend 6 | | | | |
| Milled VCZ | Jet-milled VCZ | 1 | Lactohale ® LH230 | 99 |
| Blend 1 | | | | |
| Milled VCZ | | 10 | Lactohale ® LH230 | 90 |
| Blend 2 | | | | |
| Milled VCZ | | 30 | Lactohale ® LH230 | 70 |
| Blend 3 | | | | |
| Milled VCZ | | 30 | Lactohale ® LH300 | 70 |
| Blend 4 | | | | |
| Milled VCZ | | 30 | Respitose ® SV003 | 70 |
| Blend 5 | | | | |
| Milled VCZ | | 30 | Lactohale ® LH206 | 70 |
| Blend 6 | | | | |

Scanning electron microscopy (SEM). Scanning electron microscopy (Zeiss Supra 40 C SEM, Carl Zeiss, Heidenheim an der Brenz, Germany) was used to determine the surface particle morphology of the powders made using the suspension based TFF process. A small amount of bulk powder was placed onto carbon tape. A sputter was used to coat all samples with 15 mm of 60/40 Pd/Pt before capturing the images.

Drug quantification (HPLC). The content of TAC was analyzed with an Agilent HPLC System 1220 Infinity II (Agilent, Santa Clara, Calif. USA). Two mobile phases were used in a gradient method as shown in Table 3. Mobile Phase A used 0.4% phosphoric acid in water, and Phase B used 100% acetonitrile. The absorbance of TAC was detected using UV detection at a wavelength of 215 nm. The stationary phase was a Waters XBridge C18 column (4.6 x 150 mm, 3.5 μm) (Milford, Mass., USA), and the flow rate of the mobile phase was 1.5 mL/min. The column temperature was controlled at 50° C. The retention time of TAC was approximately −12.0 min.

The content of VCZ was also analyzed with an Agilent HPLC System 1220 Infinity II (Agilent, Santa Clara, Calif., USA). A Waters Xbridge C18 column (4.6×150 mm, 3.5 μm) (Milford, Mass.) was used at a flow rate of 0.8 mL/min. The isocratic method was performed for 4 min using a mobile phase of 40:60 (% v/v) water-acetonitrile containing 0.1% (v/v) TFA. The absorbance of VCZ was detected using UV detection at a wavelength of 254 nm at 25° C. The retention time of VCZ was approximately −2.7 min.

The standard solutions of TAC were prepared in the range of 1-250 μg/mL by diluting with methanol-water (60:40, v/v). The standard solutions of VCZ were prepared in the range of 1-250 μg/mL by diluting with acetonitrile-water (50:50, v/v). All analyses maintained linearity in the range tested. All chromatography data were processed by Agilent Chemstation software (Agilent, Santa Clara, Calif., USA).

TABLE 3
HPLC gradient method of TAC

| Time | Mobile phase A | Mobile phase B | Flow rate |
|---|---|---|---|
| (min) | (%) | (%) | (mL/min) |
| 0 | 52.0 | 48.0 | 1.5 |
| 7 | 52.0 | 48.0 | 1.5 |
| 10 | 30.0 | 70.0 | 1.5 |
| 12 | 30.0 | 70.0 | 1.5 |
| 12.5 | 52.0 | 48.0 | 1.5 |
| 15 | 52.0 | 48.0 | 1.5 |

In vitro aerosol performance. Aerodynamic properties were determined using a Next Generation Pharmaceutical Impactor (NGI) (MSP Corp, Shoreview, Minn.) connected to a high-capacity pump (model HCP5, Copley Scientific, Nottingham, UK) and a critical flow controller (model TPK 2000, Copley Scientific, Nottingham, UK). An RS01 Plastiape® high-resistance inhaler (Plastiape, Osnago, Italy) was used to aerosolize the TAC dry powder, and an RS00 Plastiape® high-resistance inhaler was used to aerosolize the VCZ dry powder. These devices were attached to the induction port by a molded silicon adapter. TFF powder was dispersed into the NGI through the USP induction port at a flow rate of 60 L/min for 4 s per actuation for TAC and 58 L/min for 4.1 s for VCZ.

A pre-separator was used in this study. NGI collection plates were coated with 1.5% w/v polysorbate 20 in methanol and allowed to dry for 20 min before use. After aerosolization, the deposited powders were extracted and diluted with a mixture of water and methanol (40:60 v/v) for TAC and a mixture of water and acetonitrile (50:50 v/v) for VCZ. The content of TAC and VCZ in the deposited powders was determined using the HPLC method described in Section 2.6. Copley Inhaler Testing Data Analysis Software (CITDAS) Version 3.2 (Copley Scientific, Nottingham, UK) was used to calculate the fine particle fraction (FPF), the emitted fraction (EF), the mass median aerodynamic diameter (MMAD), and the geometric standard deviation (GSD). Both FPF and EF were calculated based on the recovered dose, which is the sum of the dose deposited on the device (capsule and device), induction port (adapter and induction port), Stages 1 through 7, and the micro-orifice collector (MOC).

X-ray powder diffraction (XRD). The crystallinity of the powders was determined using a benchtop X-ray diffraction instrument, model Miniflex 600 II (Rigaku, Tokyo, Japan), with primary monochromated radiation (Cu K radiation source, λ=1.54056 Å). The instrument was operated at an accelerating voltage of 40 kV at 15 mA. Samples were loaded in the sample holder and scanned at a scan speed of 2°/min, with a step size of 0.02° over a 2θ range of 5-40° and a dwell time of 2 s.

Specific surface area. Gas adsorption analysis was performed to determine the specific surface area (SSA) of the powders using a Monosorb rapid surface area analyzer, model MS-21 (Quantachrome, Boynton Beach, Fla., USA), as well as the single-point Braummer-Emmett-Teller (BET) method. Before the analysis, a known quantity of powder was outgassed under helium at 25° C. for 24 h. This outgassing temperature was selected to avoid heat-related degradation of the powders while still promoting water vapor removal. A mixture of nitrogen-helium (30:70 v/v) was used as the adsorbate gas. The resulting surface area was normalized by the sample weight to obtain the SSA of the powders.

Homogeneity test. Powders made using the suspension based TFF process and conventional blending were analyzed for their TAC and VCZ content, respectively, using HPLC as described in Section 2.6. Ten samples from each formulation powder were tested for the assay. Each sample weighs 20.0 mg±1 mg and diluted with methanol/water (60:40 v/v) for TAC and with acetonitrile/water (50:50 v/v) for VCZ to obtain 100 μg/mL. The percentage of content uniformity of each formulation was calculated as the percentage of TAC or VCZ to the nominal dose, while the content homogeneity of TAC and VCZ was expressed in terms of the percentage relative standard deviation (% RSD).

Degree of deagglomeration by laser diffractometer. Particle size of the TFF ordered mixture and the powder blends were measured using a HELOS laser diffractor coupled with a RODOS dry dispersion unit (Sympatec GmbH, Clausthal-Zellerfel, Germany). The sample was delivered by the rotating table at a constant rotation setting of 20%. The measurement was set to trigger every 5 ms when the optical concentration exceeded 1%. The time base was set at 100 ms, and a forced stability. The PSD of each measurement between 5% and 25% optical concentration were averaged to obtain the overall PSD. The PSD was measured in stepwise increases at the primary pressure (PP) in the range 0.25-4.0 bar. Triplicate measurement was performed at each pressure. The critical primary pressure (CPP), which is the pressure that can overcome the interactive forces holding agglomerates together, was determined using a method adapted from Jaffari et al. (Jaffari et al., 2013). The CPP was assigned when the difference in geometric median diameters between two consecutive primary pressures was lower than 6% (Jaffari et al., 2013).

Statistical analysis. The statistical significance of the EF, FPF, MMAD, and SSA of each formulation was determined using ANOVA. A p-value <0.05 was considered a significant difference. JMP 15.1 was used to compare the significance of the data.

### C. Results

1. Properties of TAC/LAC Powders Made Using the Suspension Based TFF Process

Physical properties. SEM was used to determine the surface morphology of the powders made using the suspension based TFF process. It was shown that neat LAC retained its morphology after TFF (FIG. 2). Small carriers (Lactohale® LH300, Lactohale® LH230) agglomerated, but large carriers (e.g., Respitose® SV003, Lactohale® LH206) exhibited discrete coarse particles. Fine LAC particles were found on the surface of LAC particles before and after the process.

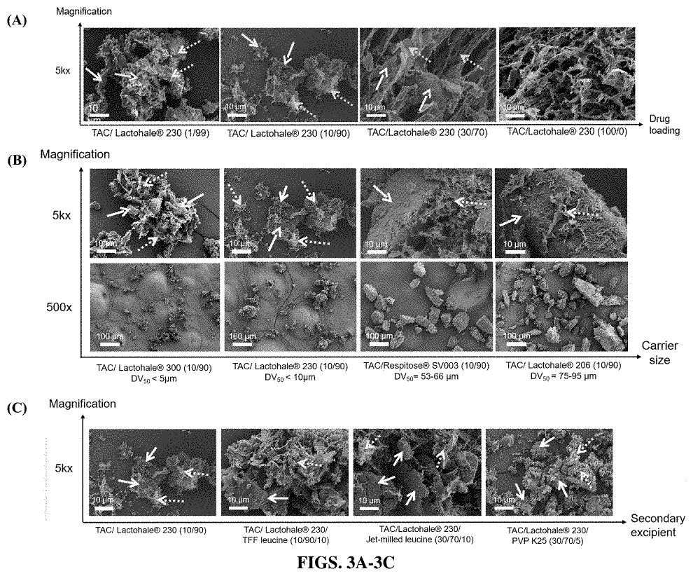

FIG. 3 shows the morphology of the TAC/LAC powders prepared using the suspension based TFF process. Nanostructured brittle matrices of TAC were found on the surface of the LAC carrier. The nanostructured brittle matrix of TAC was formed by TFF, as reported in our previous study. Drug loading resulted in a higher portion of the nanostructured brittle matrix adhering to the surface of the LAC (FIG. 3A). FIG. 3B demonstrates that the attachment of the nanostructured brittle matrix of TAC to the surface of the LAC carrier was affected by the various sizes of the LAC carrier.

The nanostructured brittle matrix of TAC and small-sized LAC are agglomerated as shown in the case of Lactohale® LH300 and Lactohale® LH230. For larger LAC carriers like Respitose® SV003 and Lactohale® LH206, we observed two particle morphologies, including the nanostructured brittle matrix of the drug and the LAC particle coated with drug aggregates. The particle size distribution of Respitose® SV003 and Lactohale® LH206 ranged from 19-106 μm and 20-170 μm, respectively (DFE Pharma, 2020). The nanostructured brittle matrix can agglomerate with small-sized LAC, but it cannot cover the surface of a large carrier, thus detaching from the carrier. Consequently, only some portion of the nanostructured aggregates attached to the surface of Respitose® SV003 and Lactohale® LH206, while other parts of the brittle matrix remained as individual brittle matrix particles. FIG. 3C demonstrates that a larger fraction of the nanostructured brittle matrix mixed with the LAC carrier after the addition of a secondary excipient.

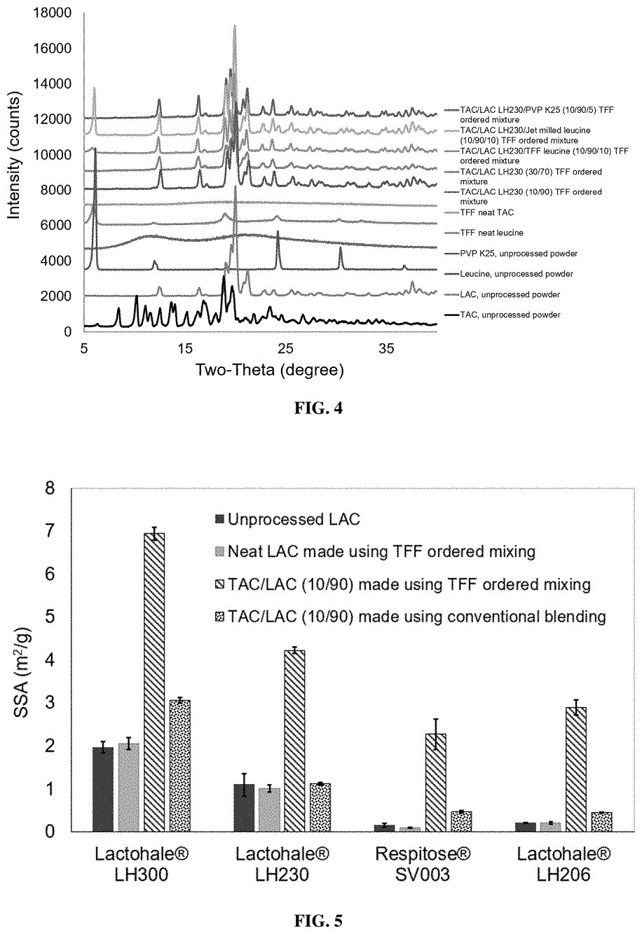

The physical state of the drug and excipient was characterized by X-ray diffraction (FIG. 4). As might be expected, peaks of the LAC carrier and jet-milled leucine were observed in the XRD diffractograms, indicating that both excipients remained crystalline after the process, since they were dispersed in the antisolvent system. The XRD diffractogram demonstrated no peak of TAC in TFF neat TAC, TAC/Lactohale® LH230 (10/90), or TFF TAC/Lactohale® LH230 (30/70). This indicates that TAC became amorphous after the process. TAC is a glass-forming ability type III drug, in which its crystallization is slow (Wyttenbach and Kuentz, 2017). This property allows the drug to remain amorphous after the process a without stabilizer. Although TFF neat leucine was prepared by dissolving leucine in water followed by TFF, the XRD diffractograms showed a peak of leucine, indicating that leucine was still crystalline after the process. After dispersing TFF leucine in the drug solution, followed by TFF, the XRD diffractograms demonstrated that TFF leucine remained crystalline, since peaks of leucine were detected in both the TFF neat leucine and the TFF mixture of TAC/Lactohale® LH230/TFF leucine (10/90/10). The addition of PVP K25 to the formulation did not affect the crystallinity of the formulation composition, since it showed that only peaks of LAC were found in TFF TAC/Lactohale® LH230/PVP K25 (10/90/5).

The specific surface area (SSA) of the powders made using the suspension based TFF process was determined by gas absorption analysis. We found no significant difference in SSA between the unprocessed LAC and TFF neat LAC (p<0.05), which indicates that the surface area of the LAC carrier was not changed by TFF. Additionally, carrier size affects the SSA of the carrier. Lactohale® LH300 exhibited the highest SSA among all four grades of LAC, while Respitose® SV003 showed the lowest SSA (FIG. 5). Despite the larger size of Lactohale® LH206, the SSA of Respitose® SV003 was smaller than that of Lactohale® LH206. This may be related to the different types of LAC Respitose® SV003 is a sieved LAC with a particle size range of 19—106 while Lactohale® LH206 is a milled LAC with a particle size range of 20-170 μm (DFE Pharma, 2020). Therefore, due to the different processes, the lower SSA of Respitose® SV003 may be related to differences in surface roughness and the amount of small LAC.

With the presence of TAC, the trend in SSA was similar to that of unprocessed LAC and TFF neat LAC. The SSA of the TAC TFF ordered mixture was ranked LH300>LH230>LH206>SV003. Similarly, the SSA of the TFF TAC containing Respitose® SV003 was smaller than that of the formulation containing Lactohale® LH206.

2. Properties of TAC/LAC Powders Made Using the Suspension Based TFF Process

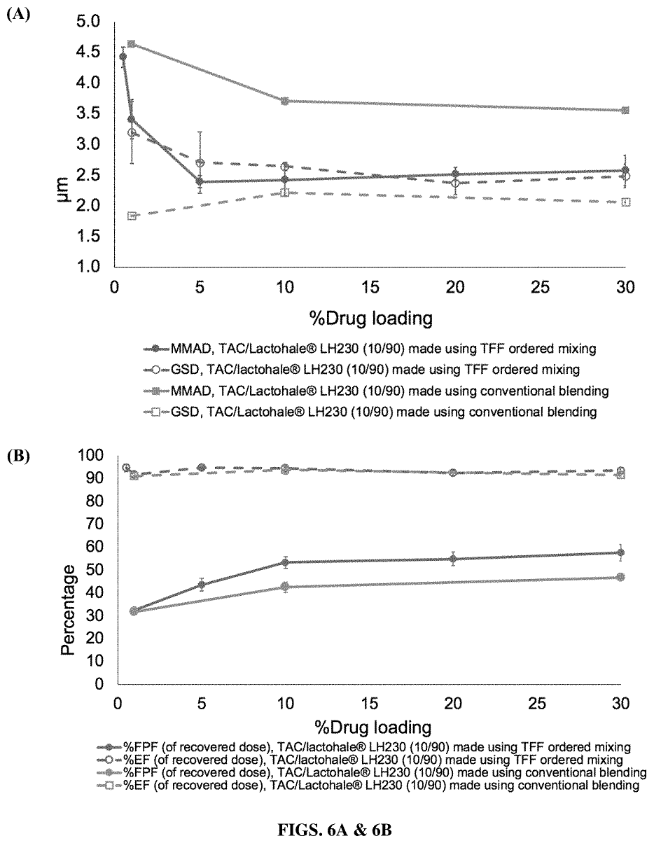

The effect of drug loading, carrier size, and the presence of a secondary excipient on the aerosol performance of TAC/LAC powders made using the suspension based TFF process was investigated. In vitro aerodynamic testing revealed that drug loading affected the aerosol performance of TAC/LAC powders made using the suspension based TFF process and conventional blending. The MMAD of TAC/LAC powders made using the suspension based TFF process significantly decreased as the drug loading was increased from the range of 1-5% (FIG. 6) (p<0.05); however, there is no significant difference in MMAD when the drug loading was in the range of 5-30%. Likewise, the fine particle fraction (FPF) of the recovered dose was significantly increased from 32% to 53% as the drug loading was increased from 1% to 10% (FIG. 6B) (p<0.05). The FPF of TAC/LAC powders made using the suspension based TFF process was consistent in the range of 53-57% as the drug loading increased from 10% to 30% (FIG. 6B). Additionally, the drug loading did not significantly affect the emitted fraction of TAC. The EF of all formulations was in the range of 91-94%.

It is important to note that the aerosol performance of TAC/LAC powders made using conventional blending also increased as the drug loading was increased. The MMAD of TAC/LAC powders made using conventional blending was significantly decreased from 4.59 μm±0.01 μm to 3.56 μm±0.01 μm as the drug loading increased from 1% to 30% (p<0.05). The FPF of the TAC/LAC (30/70) made using conventional blending was significantly higher than the TAC/LAC (1/99) made using conventional blending. Despite the similar trend, the FPF of the TAC/LAC powder made using conventional blending was smaller than the FPF of TAC/LAC powders made using the TFF suspension based TFF process over the entire drug loading range.

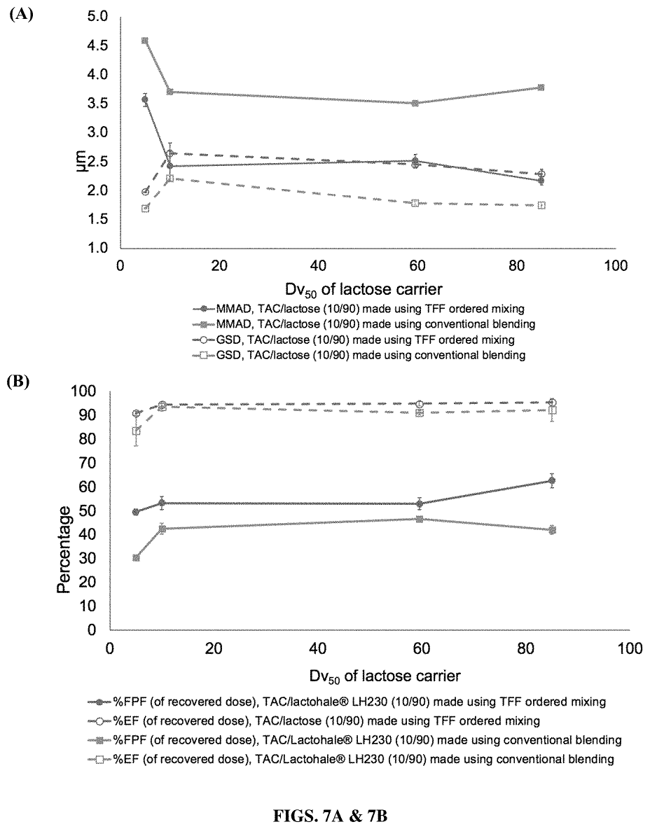

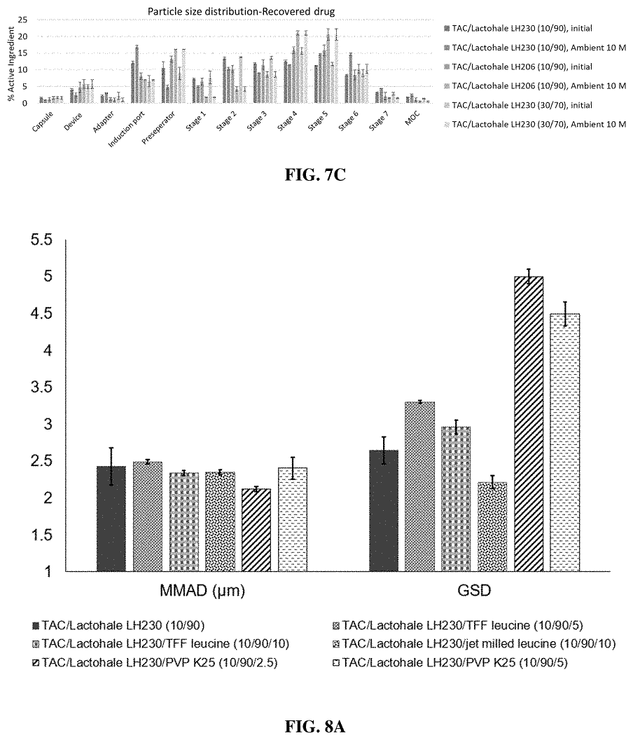

The carrier size appeared to have an impact on the aerosol performance of the TAC/LAC powders made using the suspension based TFF process and conventional blending. Both TAC/Lactohale® LH300 (10/90) made using the suspension based TFF and conventional blending showed a significantly higher MMAD and lower FPF (p<0.05) than other LAC grades (FIG. 7). Additionally, TAC/Lactohale® LH206 (10/90) made using the suspension based TFF process showed a significant smaller MMAD and higher FPF (p<0.05), compared to the other LAC grades. Finally, FIG. 7C shows the locations of the recovered drug and the percentage of the drug load that reach different penetration within the respiratory system.

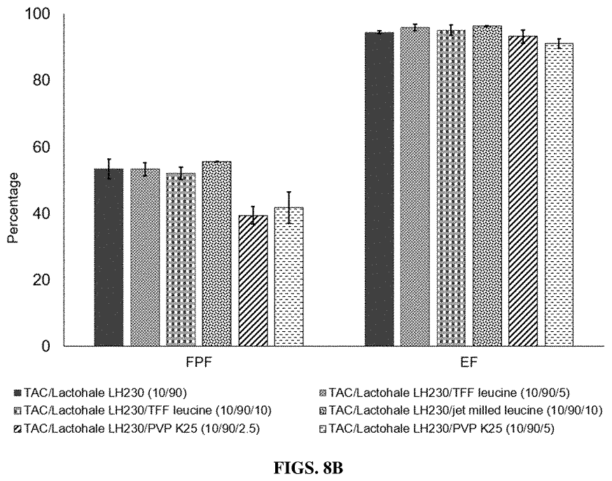

Engineered leucine particles prepared by TFF or jet milling were also used to disperse the drug from the carrier. It was found that both engineered dispersing agents appeared to have no effect on the aerosol performance of the TAC/LAC powders prepared using the suspension based TFF process. We observed no significant difference in the MMAD, FPF, and EF of formulations containing 0%, 5%, and 10% TFF leucine, which indicates that the amount of TFF leucine did not affect the aerosol performance of the TAC-LAC powders (FIG. 8). Additionally, there was no difference in the MMAD, FPF, and EF between formulations containing 10% TFF leucine and 10% jet-milled leucine, indicating that the aerosolization of the TFF powder was not affected by the morphology of leucine.

The effect of PVP K25 on the aerosol performance of the TFF powder was also investigated. In vitro aerodynamic testing demonstrated that the presence of PVP K25 decreased the aerosol performance of TAC. There was no significant difference in the MMAD, FPF, and EF between formulations containing different amounts of PVP.

3. Homogeneity of Tac/Lac Powders Made Using the Suspension Based TFF Process Versus Conventional Blending

TABLE 4
Homogeneity of TAC/LAC powders made using the suspension
based TFF process versus conventional blending. The
% RSD is the relative standard deviation and is calculated
by multiplying the standard deviation by 100 and dividing
this product by the mean. The % RSD described the spread
of the data with respect to the mean.

| | Suspension based | | | |
|---|---|---|---|---|
| | TFF process | | Conventional blending. | |
| | % Content | | % Content | |
| | (of nominal | | (of nominal | |
| Formulation | dose) | % RSD | dose) | % RSD |
| TAC/Lactohale ® | 101.5 | 2.4 | 102.6 | 17.4 |
| LH230 (1/99) | | | | |
| TAC/Lactohale ® | 100.3 | 2.7 | — | — |
| LH230 (5/95) | | | | |
| TAC/Lactohale ® | 101.4 | 2.4 | 111.3 | 5.9 |
| LH230 (10/90) | | | | |
| TAC/Lactohale ® | 100.5 | 2.5 | — | — |
| LH230 (20/80) | | | | |
| TAC/Lactohale ® | 99.9 | 3.1 | 104.3 | 13.5 |
| LH230 (30/70) | | | | |
| TAC/Lactohale ® | 98.2 | 1.4 | 107.5 | 6.2 |
| LH 300 (10/90) | | | | |
| TAC/Respitose ® | 97.2 | 2.6 | 90.1 | 21.3 |
| SV003 (10/90) | | | | |
| TAC/Lactohale ® | 99.7 | 8.1 | 101.8 | 19.5 |
| LH206 (10/90) | | | | |
| TAC/Lactohale ® | 102.0 | 2.9 | — | — |
| LH230-FF | | | | |
| leucine (10/90/5) | | | | |
| TAC/Lactohale ® | 101.2 | 1.4 | — | — |
| LH230/TFF | | | | |
| leucine (10/90/10) | | | | |
| TAC/Lactohale ® | 101.8 | 4.9 | — | — |
| LH230/jet-milled | | | | |
| leucine (10/90/10) | | | | |
| TAC/Lactohale ® | 100.4 | 1.7 | — | — |
| LH230/PVP | | | | |
| K25 (10/9/:2.5) | | | | |
| TACLactohale ® | 101.4 | 3.2 | — | — |
| LH230/PVP | | | | |
| K25 (10/90/5) | | | | |

TAC/LAC powders made using the suspension based TFF process and those made using conventional blending were analyzed to determine the uniformity of their TAC content. According to the United States Pharmacopoeia, the criterion for the content uniformity of a DPI is 85-115% of the nominal dose (Tan et al., 2019). The relative standard deviation (RSD) of 10 dosage units should less than or equal to 6% (Tan et al., 2019). HPLC analysis showed that the content of TAC was in the range of 97-102% (Table 4). The RSDs of almost all formulations made using the suspension based TFF process were generally less than 6%, except for TAC/Lactohale® LH206 (1/90). Lactohale® LH206, which had the largest carrier size, exhibited the highest RSD (8.1%), indicating higher variation than the smaller LAC carriers.

The TAC/LAC powders made using conventional blending exhibited higher variation in content uniformity than powders made using the suspension based TFF process. The content of TAC varied from 90-111% of the nominal dose. The RSD of TAC was about 6-21% RSD. Interestingly, the smaller size of LAC showed smaller RSD than the larger size of LAC. The RSD of TAC/Lactohale® LH 300 (10/90) and TAC/Lactohale® LH230 (10/90) was about 6%, while the RSD of TAC/Respitose® (10:90) and TAC/Lactohale® LH206 (10/90) were 21.3 and 19.5, respectively.

4. Critical Primary Pressure of TAC/LAC Powders Made Using the Suspension Based TFF Process Versus Conventional Blending

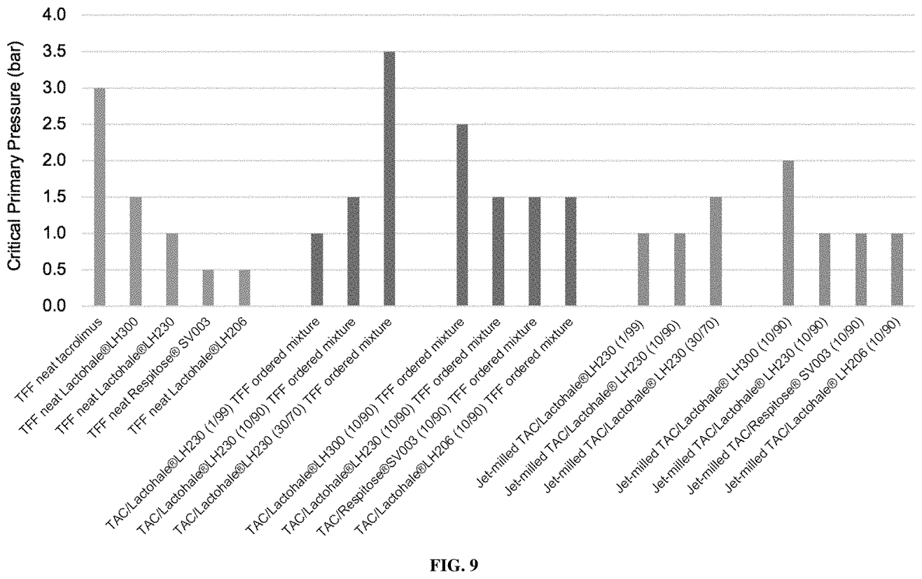

The degree of deagglomeration of the powders was determined by dry dispersion laser diffractometry adopted from Jaffari's study (Jaffari et al., 2013). The critical primary pressure is the pressure at which particle size reaches a plateau, which indicates the dispersing pressure required to overcome the interactive forces holding agglomerates together (Jaffari et al., 2013). The CPP also represents the cohesivity of the powder and the degree of powder deagglomeration (Jaffari et al., 2013). FIG. 9 shows the CPP of powders made using the suspension based TFF process and conventional blending. It was demonstrated that the CPP of the powders made using the suspension based TFF process and conventional blending was affected by the drug loading of TAC. For the suspension based TFF process, the CPP of TAC/Lactohale® LH230 (30/70) was 2.5 bar and 0.5 bar higher than TAC/Lactohale® LH230 (10/90) and TAC/Lactohale® LH230 (1/99), respectively (FIG. 9). This indicates that higher drug loading resulted in a lower degree of deagglomeration.

A similar trend was found in TAC/Lactohale® LH230 made using conventional blending. The CPP of TAC/Lactohale® LH230 (30/70) made using conventional blending was 0.5 bar higher TAC/Lactohale® LH230 (10/90) and TAC/Lactohale® LH230 (1/99). Interestingly, the TAC/LAC powders made using the suspension based TFF process exhibited higher CPP than the TAC/LAC powders made using conventional blending (FIG. 9). Only TAC/Lactohale® LH230 (1/99) made using the suspension based TFF process exhibited the same CPP as TAC/Lactohale® LH230 (1/99) made using conventional blending.

Additionally, carrier size affected the deagglomeration of the powders. For neat LAC powders made using the suspension based TFF process, the larger particle size of LAC resulted in lower CPP (FIG. 9), indicating that the larger particle size of LAC exhibited more deagglomeration. This is consistent with the trends observed in the TAC/LAC powders made using both the suspension based TFF process and conventional blending. TAC/Lactohale® LH300 (10:90) made using both methods showed higher CPP than other formulations containing larger sizes of LAC. Despite the same formulation compositions, the TAC/LAC powders made using the suspension based TFF process exhibited a higher CPP than powders made using conventional blending.

Interestingly, different types of secondary excipients have an impact on the degree of deagglomeration of the TFF powders. The blue bars in FIG. 9 show the comparison of the CPP of formulations containing secondary excipients made using the suspension based TFF process. Only TAC/Lactohale® LH230/TFF leucine (10/90/10) exhibited higher CPP than TAC/Lactohale® LH230 (10/90). The CPP of TAC/Lactohale® LH230/jet-milled leucine (10/90/10) and TAC/Lactohale® LH230/PVP K25 (10/90:5) were similar to that of TAC/Lactohale® LH230 (1/90), indicating that the addition of jet-milled leucine and PVP K25 did not affect the degree of deagglomeration in the TFF ordered mixture powder.

5. Properties of VCZ-LAC Powders Made Using the Suspension Based TFF Process

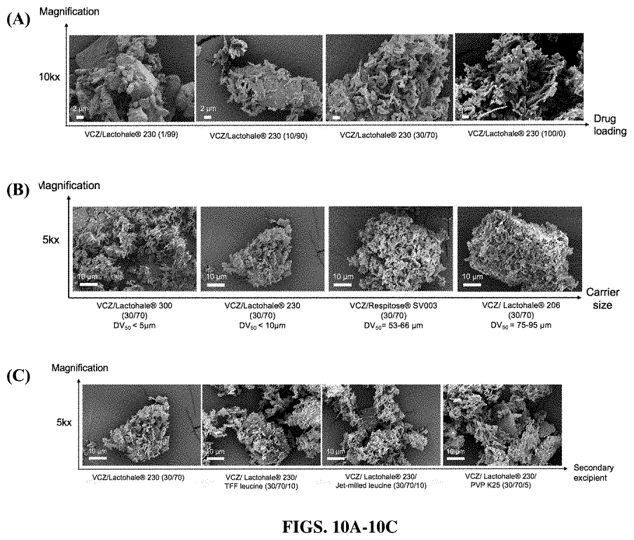

Physical properties. FIG. 10 shows the particle morphology of VCZ/LAC powders prepared using the suspension based TFF process. VCZ formed nanoaggregates on the surface of the LAC carrier. FIG. 10A demonstrates that higher drug loading of VCZ resulted in a larger portion of nanoaggregates on the LAC carrier. The particle size of LAC appeared to have an effect on the particle morphology of the TFF ordered mixture. FIG. 10B shows that small LAC carriers such as Lactohale® LH300 and Lactohale® LH230 were agglomerated with VCZ nanoaggregates, while larger carriers such as Respitose® SV003 and Lactohale® LH206 exhibited discrete particles covered by VCZ nanoaggregates. Similar to the TAC case, PVP and TFF leucine formed brittle matrices after TFF, resulting in the attachment of brittle matrices on the LAC carrier (FIG. 10C).

FIG. 11 shows the crystallinity of VCZ and excipients made using the suspension based TFF process. Peaks of VCZ were observed at ˜13.5° and 17.5° in both TFF VCZ/Lactohale® LH230 (30/70) and TFF neat VCZ, indicating that VCZ was crystalline after TFF. Additionally, LAC, jet-milled leucine, and TFF leucine, which were dispersed in the antisolvent system, exhibited sharp peaks in the XRD diffractograms. This indicates that both LAC and leucine remained crystalline after the process. The addition of PVP K25 did not affect the crystallinity of VCZ. Peaks of VCZ were also observed in VCZ/Lactohale® LH230/PVP K25 (30/70/5).

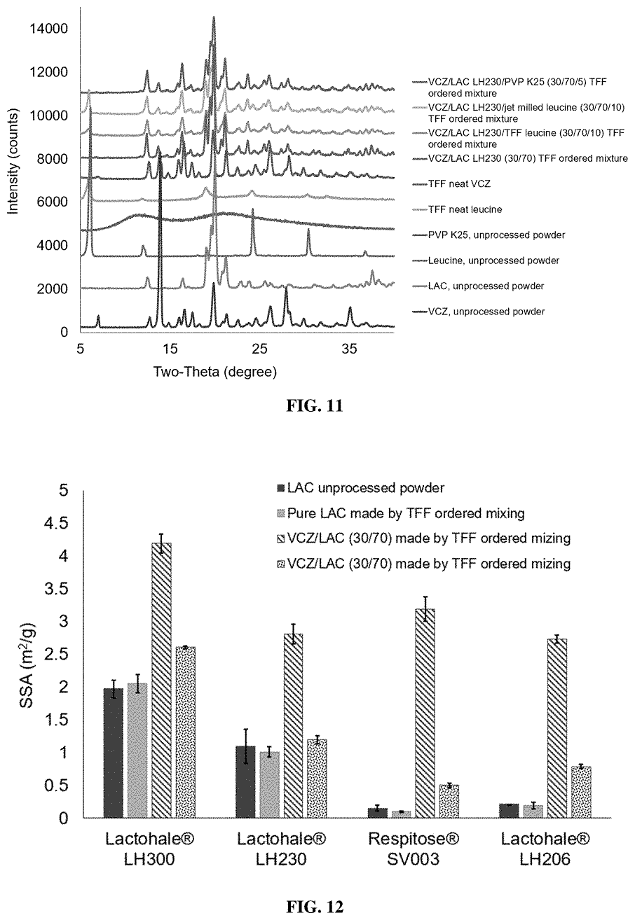

With the presence of VCZ, there were significant increases in SSA over the TFF neat LAC (p<0.05). VCZ/Lactohale® LH300 made using the suspension based TFF process exhibited the highest SSA among the LAC grades; however, no significant differences in SSA were observed between the other grades of LAC (FIG. 12). It was shown that VCZ/LAC powders made using conventional blending exhibited trends similar to LAC unprocessed powders and neat LAC. The SSA of VCZ/LAC powders made using conventional blending was ranked as follows: Lactohale® LH300>Lactohale® LH230>Lactohale® LH206>Respitose®5V003.

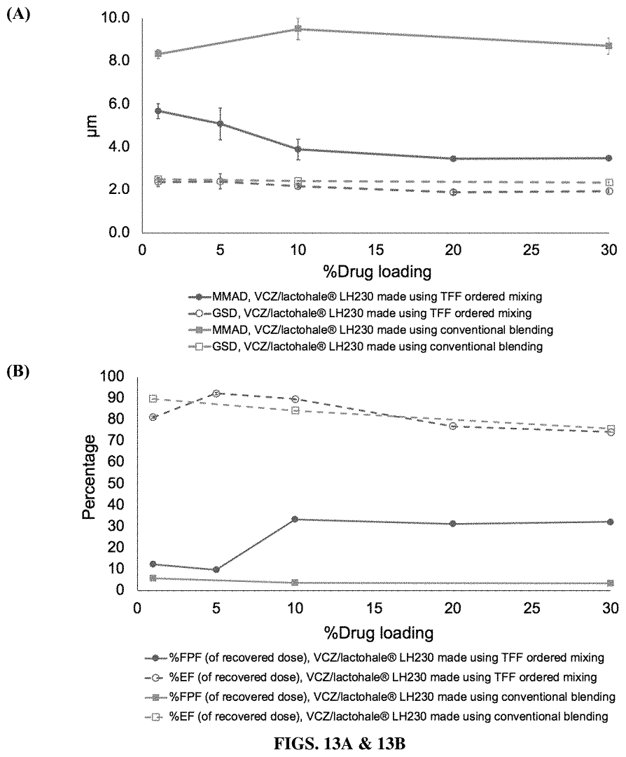

As in the TAC case, we investigated the effect of drug loading, carrier size, and the presence of a secondary excipient on the aerosol performance of VCZ. Drug loading affected the aerosol performance of VCZ/LAC powders made using the suspension based TFF process. The in vitro aerodynamic testing demonstrated that MMAD significantly decreased from 5.68 μm±0.36 μm to 3.90 μm±0.48 μm as the drug loading in the TFF formulations was increased from 1% to 10% (FIG. 13A) (p<0.05). There was no significant difference in MMAD when the drug loading exceeded 10%. Likewise, the FPF of VCZ/LAC powders made using the suspension based TFF process increased from 12.38%±1.98% to 33.21%±5.17% when the drug loading was increased from 1% to 10% (FIG. 13B). The FPF did not change when the drug loading exceeded 10% (FIG. 13B). This contrasts with the VCZ/LAC powders made using conventional blending. Drug loading did not significantly affect the aerosol performance of VCZ/LAC powders made using conventional blending. For conventional blending, the FPF of VCZ-Lactohale® LH230 (1:99) was slightly higher than VCZ/Lactohale® LH230 (30/70); however, there was no significant difference in MMAD between VCZ/Lactohale® LH230 (1/99) and VCZ/Lactohale® LH230 (30/70).

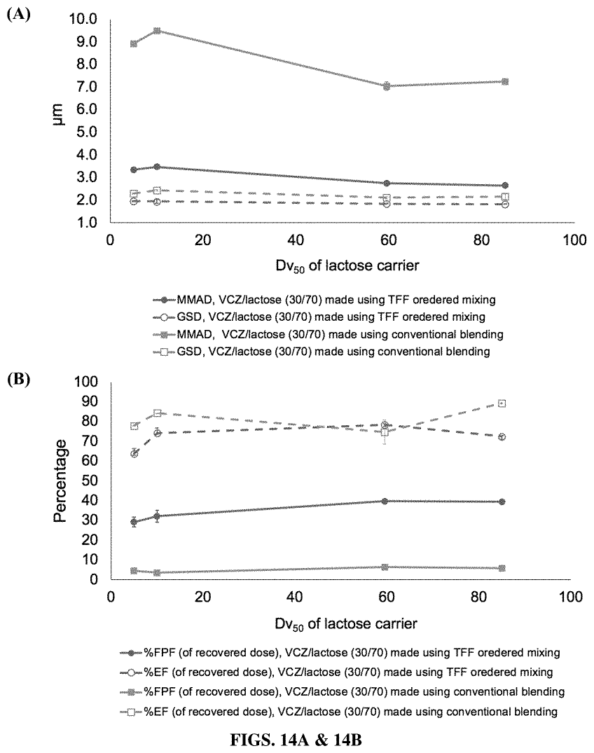

Carrier size had an impact on the aerosol performance of VCZ/LAC powders made using the suspension based TFF process and powders made using conventional blending. It was shown that VCZ/Respitose® SV003 (30/70) and VCZ/Lactohale® LH206 (30/70) made using the suspension based TFF process exhibited significantly higher FPF and smaller MMAD than the other grades of LAC (p<0.05) (FIG. 14). Likewise, VCZ/Respitose® SV003 (30/70) and VCZ/Lactohale® LH206 (30/70) made using conventional blending exhibited lower MMAD and higher FPF than other larger LAC sizes (FIG. 14). These results indicate that the larger size of LAC resulted in better aerosol performance.

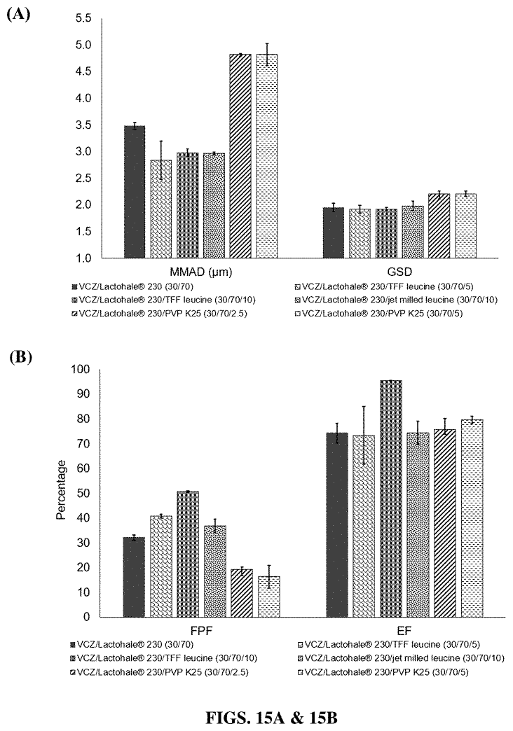

The addition of a secondary excipient appears to have an impact on the aerosol performance of VCZ/LAC powders made using the suspension based TFF process. Similar to the case of TAC, the presence of PVP K25 decreased the aerosol performance of VCZ, as it showed a significant increase in MMAD and a significant decrease in FPF (p<0.05) (FIG. 15). Additionally, the addition of TFF leucine and jet-milled leucine increased the aerosol performance of VCZ/LAC powder. The formulation containing jet-milled leucine and TFF leucine exhibited significantly smaller MMAD and higher FPF than the formulation without leucine (p<0.05). Interestingly, the formulation containing 10% TFF leucine showed similar MMAD, but higher FPF and EF than the formulation containing 10% jet-milled leucine. This indicates that TFF leucine appears to have better dispersing properties than jet-milled leucine.

6. Homogeneity of VCZ/Lac Powders Made Using the Suspension Based TFF Process Versus Conventional Blending.

TABLE 5
Homogeneity of VCZ/LAC powders made using the suspension
based TFF process versus conventional blending.

| | Suspension based | | | |
|---|---|---|---|---|
| | TFF process | | Conventional blending | |
| | % Content | | % Content | |
| | (of nominal | | (of nominal | |
| Formulation | dose) | % RSD | dose) | % RSD |
| VCZ/Lactohale ® | 102.8 | 6.8 | 106.7 | 14.6 |
| LH230 (1/99) | | | | |
| VCZ/Lactohale ® | 97.4 | 0.6 | — | — |
| LH230 (5/95) | | | | |
| VCZ/Lactohale ® | 97.8 | 0.4 | 120.6 | 14.4 |
| LH230 (10/90) | | | | |
| VCZ/Lactohale ® | 96.4 | 3.2 | — | — |
| LH230 (20/80) | | | | |
| VCZ/Lactohale ® | 99.9 | 1.1 | 114.2 | 12.3 |
| LH230 (30/70) | | | | |
| VCZ/Lactohale ® | 101.4 | 0.9 | — | — |
| LH230 (40/60) | | | | |
| VCZ/Lactohale ® | 100.9 | 0.5 | 96.6 | 9.5 |
| LH300 (30/70) | | | | |
| VCZ/Respitose ® | 101.8 | 2.0 | 92.3 | 7.6 |
| SV003 (30/70) | | | | |
| VCZ/Lactohale ® | 102.5 | 8.5 | 96.6 | 14.6 |
| LH206 (30/70) | | | | |
| VCZ/Lactohale ® | 100.5 | 1.4 | — | — |
| LH230/TFF | | | | |
| leucine(30/70/5) | | | | |
| VCZ/Lactohale ® | 99.1 | 1.1 | — | — |
| LH230/TFF | | | | |
| leucine(30/70/10) | | | | |
| VCZ/Lactohale ® | 102.2 | 5.7 | — | — |
| LH230/jet-milled | | | | |
| leucine (30:/70/10) | | | | |
| VCZ/Lactohale ® | 97.5 | 1.2 | — | — |
| LH230/PVP K25 | | | | |
| (30/70/2.5) | | | | |
| VCZ/Lactohale ® | 100.1 | 1.2 | — | — |
| LH230/PVP K25 | | | | |
| (30/70/5) | | | | |

The content uniformity of VCZ/LAC powders made using the suspension based TFF process and powders made using conventional blending were analyzed by HPLC. The content of VCZ in the powders made using the suspension based TFF process was in the range of 96-102.5% of the nominal dose (Table 5). Similar to the case of TAC, the RSDs of almost all TFF formulations were generally less than 6%, except for VCZ/Lactohale® LH230 (1/99) and VCZ/Lactohale® LH206 (30:70) made using the suspension based TFF process. VCZ/Lactohale LH206 (30/70) exhibited the largest RSD (8.1%), indicating the highest variation.

Despite the same formulation compositions, the VCZ/LAC powder made using the suspension based TFF process demonstrated more variation than powders made using conventional blending. The content of VCZ in all powder blends varied from 92.3-120.6% of the nominal dose. The RSDs of VCZ were in the range of 7.6-14.6%. VCZ/Lactohale® LH206 (30/70) made by conventional blending exhibited higher RSD (14.6%) than other LAC grades. Moreover, the drug loading appeared to have no effect on the homogeneity of the powder blend. The RSDs of VCZ/Lactohale® LH230 made by conventional blending containing different drug ratios were all higher than 12%.

7. Critical Primary Pressure of VCZ/LAC Powders Made Using the Suspension Based TFF Process Versus Conventional Blending.

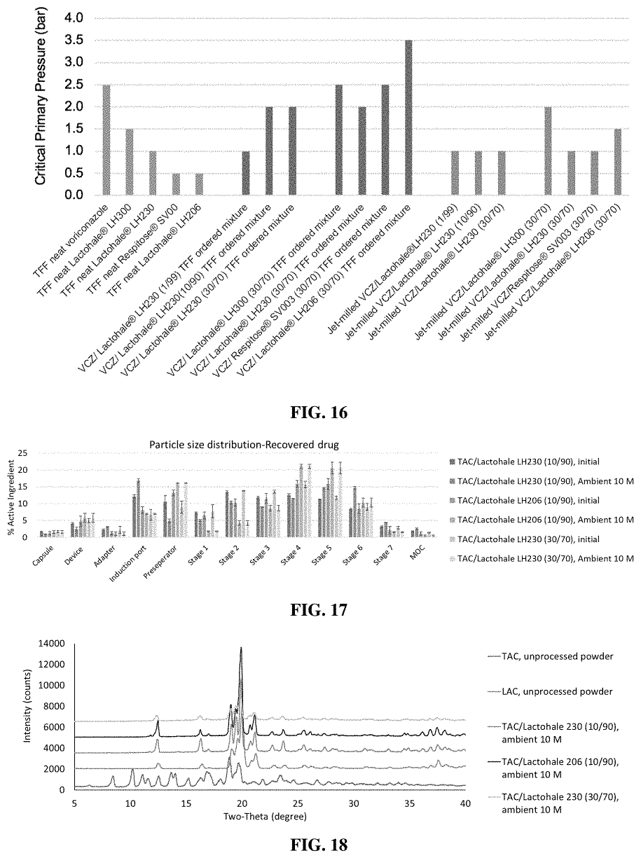

The CPP of powders made using the suspension based TFF process and conventional blending was determined by laser diffraction. FIG. 16 shows that drug loading affected the CPP of VCZ/LAC powder made using the suspension based TFF process, but it did not affect the CPP of VCZ/LAC powders made using conventional blending. Higher drug loading in the VCZ/LAC powder made using the suspension based TFF process led to higher CPP, indicating that VCZ/LAC powders containing higher drug loading exhibited less deagglomeration. Conversely, the CPP of the VCZ/LAC powders made using conventional blending did not change as the drug loading was increased. The VCZ/LAC powders containing 10% and 30% drug loading made using the suspension based TFF process exhibited higher CPP than powder made using conventionally blending with the same compositions. This indicates that the degree of deagglomeration in the powders made using the suspension based TFF process was lower than that of powders made using conventional blending.

The carrier size also affected the CPP of powders made using the suspension based TFF process and powders made using conventional blending. Although, the CPP of VCZ/Lactohale® LH300 (30:70) showed higher CPP than that of VCZ/Lactohale® LH230 (30/70), a larger size of LAC generally resulted in higher CPP, compared to fine LAC grades.

The addition of a secondary excipient increased the CPP of VCZ/LAC powders made using the suspension based TFF process. This contrasts with the case of TAC. Higher content of secondary excipient resulted in higher CPP, indicating that the degree of deagglomeration was decreased by the addition of TFF leucine, jet-milled leucine, and PVP K25.

8. Blend Uniformity of 10% Tacrolimus Blend Prepared by the Conventional Blending of TFF TAC/LAC (50/50) with Lactose Inhalation Grade.

A. Blend Preparation Procedure:

10% Tacrolimus Blend, Lot 19TF105, was prepared by blending 20 grams of 50% Tacrolimus Blend (Lot 19TF078) with 80 grams of Lactose (Respitose SV-003) in a V-Blender. The blend was mixed by adding 50% Tacrolimus to the 80 grams of Lactose in 5-gram increments, and then blending for 15 minutes. After the final 50% Tacrolimus blend was added to the V-blender, the blend was mixed for 30 minutes (75 minutes of blending total).

B. Blend In-Process Uniformity:

After blending, five 15-25 mg samples were collected from the blend using a plastic spatula and then analyzed by weighing 15 mg for each sample and diluting in 5 mL of diluent (50:50 Water:ACN) (0.3 mg/mL concentration). The average of the 5 samples was 101.6% and samples had a recovery range of 96.7%-104.3% (Table 7).

TABLE 7
19TF105 Blend Uniformity In-Process Results
In-process Blend Uniformity

| | | Concen- | | | |
|---|---|---|---|---|---|
| | Peak | tration | | | |
| Sample | Area | (mg/mL) | Recovery | Criteria | Result |
| QC-7929CL-1 | 1588.7 | 0.31 | 103.4% | ±5% of | PASS |
| QC-7929CL-2 | 1611.8 | 0.31 | 104.3% | Average | PASS |
| QC-7929CL-3 | 1541.9 | 0.30 | 101.1% | | PASS |
| QC-7929CL-4 | 1578.1 | 0.31 | 102.8% | | PASS |
| QC-7929CL-5 | 1465.4 | 0.29 | 96.7% | | PASS |
| | Average | 0.30 | 101.6% | 95%-105% | PASS |

Additionally, the assay testing of lot 19TF105 was performed in duplicate. The first set of duplicates had a percent recovery of 93.2% and 83.9%, this failed the stated criteria of NMT 5.0% difference between samples (Table 8). A second set of duplicates was produced had a recovery of 86.2% and 86.4%. These samples failed the Final Product specification of 90-110% recovery of Tacrolimus.

TABLE 8
19TF105 Release Assay

| | | % difference | |
|---|---|---|---|
| Sample | Recovery | between samples | Result |
| QC-7936CL-1 | 93.2% | NMT 5.0% | 9.3% |
| QC-7936CL-2 | 83.9% | | FAIL |
| QC-7936CL-1 Repeat | 86.2% | NMT 5.0% | 0.2% |
| QC-7936CL-2 Repeat | 86.4% | | PASS |

86.3%

The content uniformity of capsules that were filled with 10% tacrolimus blend were tested. 30 capsules were filled with 5 mg of 10% Tacrolimus powder, lot 19TF105, by individually weighing each capsule and then 10 capsules were sampled to be analyzed via HPLC for content uniformity. The samples were diluted to 5 mL in a 1:4 DI Water:DMSO mixture (0.1 mg/mL). The samples failed the first test for USP <905> with an AV value of 30.1 (Table 9).

TABLE 9
Content Uniformity for hand filled 10% Tacrolimus Capsules.

| | mg API | |
|---|---|---|
| Sample | recoveryed | % Recovery |
| capsule# 1 | 0.46 | 92.96 |
| capsule# 2 | 0.42 | 84.84 |
| capsule# 3 | 0.41 | 81.92 |
| capsule# 4 | 0.39 | 78.26 |
| capsule# 5 | 0.30 | 59.79 |
| capsule# 6 | 0.53 | 106.33 |
| capsule# 7 | 0.39 | 78.16 |
| capsule# 8 | 0.40 | 80.99 |
| capsule# 9 | 0.48 | 95.28 |
| capsule# 10 | 0.39 | 77.38 |
| | 0.44 | 83.59 |
| | SD | 12.54 |
| | AV | 30.1 |
| | Result | FAIL |
| | (Criteria ≤15.0) | |

After the failure of capsules for content uniformity, the blend potency and capsule filling process were tested. The blend potency was tested by diluting 150 mg of 10% Tacrolimus blend in 5 mL of 1:4 DI Water:DMSO diluent (3 mg/mL). The potency was found to be 110.3% of the Label Claim (Table 10). To test if the capsules were potentially interfering with the assay results or if the filling of the blend directly into capsules was the issue, 5 mg of blend was filled directly in a 5 mL volumetric flask and then a capsule was added into the flask. The material was then dissolved in a 1:4 mixture DI Water:DMSO diluent (0.1 mg/mL concentration). Direct filling into the capsule resulted in an assay range of 95.0% to 106.3% (Table 11).

TABLE 10
Potency Assay of 19TF105

| | mg API | |
|---|---|---|
| Sample | recovered | % of TAC |
| Assay | 17.01 | 110.3% |

TABLE 11
Recovery of 10% Tacrolimus Blend in the presence of capsules.

| Sample | % of TAC |
|---|---|
| 1 | 95.0% |
| 2 | 106.3% |
| 3 | 100.7% |

To help increase the accuracy of filling 10% Tacrolimus directly into capsules, it was decided to add an ionizer bar to the balance to help limit static interference during the weighing process. An additional 10 capsules were then filled. The samples for HPLC were processed in the same manner as the first content uniformity samples (1:4 DI Water:DMSO diluent). With the addition of the Ionizer Bar, the samples content uniformity improved, but the average recovery was still lower (Table 12).

TABLE 12
Second Content Uniformity of capsules
filled with 10% Tacrolimus Blend.

| Sample | % Recovery |
|---|---|
| capsule# 1 | 93.3% |
| capsule# 2 | 80.3% |
| capsule# 3 | 73.8% |
| capsule# 4 | 86.7% |
| capsule# 5 | 82.0% |
| capsule# 6 | 86.8% |
| capsule# 7 | 80.3% |
| capsule# 8 | 84.6% |
| capsule# 9 | 78.4% |
| capsule# 10 | 87.5% |

The 10% tacrolimus blend powder was filled in bottles. An unopened bottle of 10% tacrolimus powder was sampled for uniformity taking a top, middle, and bottom sample. After taking these samples, all of the remaining powder in the bottle was then dissolved with the 1:4 DI Water:DMSO solution and quantitatively transferred to a 200-mL volumetric flask. The uniformity samples showed a clear stratification of the API in the bottle, with the % of API found decreasing the lower in the bottle you sampled (Table 13). The whole bottle assay of 100.2% showed that the API was not lost during the filling of the bottles with powder. Based on this initial result, it was believed that separation of the Lactose and Tacrolimus TFF powder was occurring, with the denser lactose powder settling down over time.

TABLE 13
Blend uniformity and assay for unopened bottles

| | Sample | | | | |
|---|---|---|---|---|---|
| 19TF105 | Size | mg (API) | | | |
| Bottle #7 | (mg) | found | % In Blend | % of LC | Average |
| Top | 14.3 | 1.91 | 13.3% | 133.5% | 105.3% |
| Middle | 13.4 | 1.29 | 9.6% | 96.3% | |
| Bottom | 15.6 | 1.34 | 8.6% | 86.3% | |
| Whole | 5050 | 505.95 | 10.0% | 100.2% | |
| Bottle | | | | | |

Based on the individual bottle showing separation of blend uniformity with minimal handling, it was determined that uniformity among the filled bottles needed to be tested to determine if separation of the blend was occurring during the filling process. Bottles #1, #11, and Bottle #18 were assayed by dissolving all powder in the bottle using the 1:4 DI Water:DMSO solution and quantitatively transferred to 200-mL volumetric flasks. During the filling process the potency of 50% Tacrolimus Powder in each bottle decreased. The first bottle had an assay of 111.5% of the label claim, the middle bottle (#11) had an assay of 101.9% and the end bottle (#18) had an assay of 92.7% (Table 14).

TABLE 14
Uniformity of tacrolimus in filled bottles.

| | Sample | | | |
|---|---|---|---|---|
| 19TF105 | Size | mg (API) | | |
| Bottle | (mg) | found | % In Blend | % of LC |
| #1 | 5170.00 | 576.34 | 11.1% | 111.5% |
| #11 | 5180.00 | 527.83 | 10.2% | 101.9% |
| #18 | 4910.00 | 454.93 | 9.3% | 92.7% |

To restore the uniformity of tacrolimus, tacrolimus blend powder in a bottle was inverted by hand for 20 times and sampling for assay test, and then inverting the bottle an additional 20 times and sampling the bottle for a third time. Inverting the powder 20 or 40 times in a plastic bottle did not seem to improve blend uniformity as the RSD between samples did not improve (Table 15).

TABLE 15
Uniformity of 10% tacrolimus powder in plastic bottles after inversion

| | | Sample | | | | |
|---|---|---|---|---|---|---|
| | | Size | mg (API) | | | Average ± |
| 19TF105 Bottle #8 | | (mg) | found | % In Blend | % of LC | RSD |
| No | Top | 15.32 | 1.72 | 11.2% | 112.0% | 101.9% ± |
| Inversions | Middle | 17.11 | 1.70 | 9.9% | 99.4% | 8.9% |
| | Bottom | 16.64 | 1.57 | 9.4% | 94.4% | |
| 20 | Top | 12.73 | 1.03 | 8.1% | 81.2% | 89.6% ± |
| inversions | Middle | 16.58 | 1.54 | 9.3% | 92.9% | 8.1% |
| | Bottom | 15.99 | 1.51 | 9.5% | 94.5% | |
| 40 | Top | 18.12 | 2.15 | 11.9% | 118.8% | 98.7% ± |
| inversions | Middle | 16.30 | 1.45 | 8.9% | 89.2% | 17.6% |
| | Bottom | 17.24 | 1.52 | 8.8% | 88.1% | |

To try to reduce the static build up in the powder, if 10% blends will be stored in glass bottles instead of plastic. To see if inverting by hand in a glass bottle could return uniformity, powder from bottle #8 was transferred from its plastic bottle to a glass bottle. The bottle was sampled, tapped 200 times, and then sampled again. The tapping was done to see if forced segregation of powder can be achieved in case the process of transferring the powder from the plastic bottle to a glass bottle helped re-establish uniformity. After tapping, the bottle was inverted 50 times and then an additional 50 times with sampling occurring after each mixing interval. Transferring from the plastic bottle to a glass bottle increased dis-uniformity of the powder and tapping further settled the lactose and increased the concentration of the 50% Tacrolimus TFF powder at the top of the bottle (Table 16). The top sample for the tapped bottle had a concentration of 349% of the target concentration. Inverting by hand was able to restore some uniformity of the sample after tapping but was unable to restore uniformity to what would be an acceptable level.

TABLE 16
Uniformity of 10% tacrolimus powder in glass bottles after inversion

| | | Sample | | | | |
|---|---|---|---|---|---|---|
| | | Size | mg (API) | | | Average ± |
| 19TF105 Bottle #8 | | (mg) | found | % In Blend | % of LC | RSD |
| No | Top | 16.95 | 2.60 | 15.3% | 153.3% | 107.3% ± |
| Inversions | Middle | 15.62 | 1.20 | 7.7% | 76.8% | 37.8% |
| | Bottom | 13.28 | 1.22 | 9.2% | 91.8% | |
| After | Top | 12.16 | 4.24 | 34.9% | 349.0% | 168.0% ± |
| Tapping | Middle | 15.13 | 1.38 | 9.1% | 91.3% | 93.7% |
| 200 times | Bottom | 14.93 | 0.95 | 6.4% | 63.6% | |
| 50 | Top | 14.45 | 2.10 | 14.5% | 145.2% | 104.1% ± |
| inversions | Middle | 13.37 | 1.25 | 9.3% | 93.5% | 35.5% |
| | Bottom | 15.33 | 1.13 | 7.4% | 73.6% | |
| 100 | Top | 15.12 | 0.93 | 6.2% | 61.7% | 85.9% ± |
| inversions | Middle | 12.99 | 1.00 | 7.7% | 77.0% | 34.5% |
| | Bottom | 15.93 | 1.90 | 11.9% | 119.0% | |

After inverting by hand was unable to re-establish blend uniformity, it was decided to try to roll the bottles to see if a longer rolling time could be used to bring back uniformity. A new plastic bottle (bottle #10) was rolled for 30 minutes at 35 ROMs and the glass bottle from Test #4 was rolled for 30 minutes at 70 RPM. Both bottles were then sampled for uniformity. While the plastic bottle did not become uniform after rolling, it appears the glass bottle did (Table 17). The glass bottle had an assay of 94.8% and the relative standard deviation was 3.1%.

TABLE 17
Uniformity of 10% Tacrolimus Powder after rolling of bottles

| | | Sample | | | | |
|---|---|---|---|---|---|---|
| 19TF105 10% | | Size | mg (API) | | | Average ± |
| Tacrolimus Powder | | (mg) | found | % In Blend | % of LC | RSD |
| Plastic | Top | 14.9 | 1.02 | 6.8% | 68.3% | 85.5% ± |
| (Bottle #10) | Middle | 14.3 | 1.08 | 7.6% | 75.8% | 27.6% |
| | Bottom | 14.3 | 1.61 | 11.2% | 112.3% | |
| Glass | Top | 15.9 | 1.56 | 9.8% | 97.9% | 94.8% ± |
| (Bottle #8) | Middle | 12.7 | 1.17 | 9.2% | 92.0% | 3.1% |
| | Bottom | 15.5 | 1.47 | 9.5% | 94.5% | |

One potential thought to avoid issues of powder segregation in the bottles was to use the v-blender to blend the 10% Tacrolimus Powder and then fill directly into capsules from the V-blender using a fill gun. This fill gun uses vacuum to suck powder up into a dosing gun and then can be dispensed into capsules by releasing the vacuum. As we know handling the powder decreases uniformity, the blend could then be repeatedly blended in the V-blender as needed during capsule filling. For this study, 4 bottles of 19TF105 were added to the V-blender and blended for 30 minutes. The powder was then sampled for uniformity before dosing 20 doses of 15 mg (3 times the target fill weight) with the fill gun (dose 1, 10, and 20 were collected in 5 mL vials and dissolved in diluent).

Using the V-blender, we were unable to restore uniformity to the 10% blend (Table 18). The inability to establish uniformity in the V-blender could have been the result of a couple issues. V-blenders are usually meant to be operated within a certain volume capacity of the v-blender; 20 grams of 10% tacrolimus powder occupied a small volume within the V-blender and might not have had enough volume for good blending to occur. It was also observed after blending that more powder was sticking to the sides of the V-blender walls than during the initial GMP manufacturing. This extra powder sticking to the walls could have been the result of extra static charge generated transferring the powder from the bottle to the V-blender or because with a lower volume in the V-blender the powder is moving more and generating more static charge. This extra charge could impact the ability to create a uniform blend.

TABLE 18
Blend uniformity of 19TF105 10%
Tacrolimus powder in V-blender.

| | Sample | | | | |
|---|---|---|---|---|---|
| 19TF105 | Size | mg (API) | | | Average ± |
| Powder | (mg) | found | % In Blend | % of LC | RSD |
| Top | 13.8 | 1.55 | 11.2% | 112.5% | 71.8% ± |
| Middle | 17.2 | 0.78 | 4.5% | 45.4% | 49.7% |
| Bottom | 14.9 | 0.86 | 5.8% | 57.6% | |

The Fill Gun exhibited less uniformity than the V-blend and showed a bias to the 50% Tacrolimus powder within the blend (Table 19). This is likely because of the better aerosol properties of the 50% Tacrolimus blend then the Respitose lactose the 10% blend is made with.

TABLE 19
Dosing of 19TF105 with Fill Gun

| | Sample | | | | |
|---|---|---|---|---|---|
| 19TF105 | Size | mg (API) | | | Average ± |
| Powder | (mg) | found | % In Blend | % of LC | RSD |
| Top | 19.9 | 1.34 | 6.7% | 67.3% | 163.8% ± |
| Middle | 8.6 | 2.25 | 26.2% | 262.0% | 59.4% |
| Bottom | 13.4 | 2.17 | 16.2% | 162.2% | |

9. Compositions after Storage

In order to determine the effects of storage upon the composition, samples of the compositions were left stored at ambient conditions for approximately 10 months. These compositions were compared to their initial properties to determine if there was any substantial change in aerosol performance. The relevant properties are shown below in Table 20. A distribution profile of the compositions into the respiratory system can be found in FIG. 17. Similarly, the crystallinity or lack thereof the tacrolimus composition after 10 months is shown in FIG. 18. After 10 months, these compositions remained amorphous.

TABLE 20
Analysis of Composition After Storage at Ambient Conditions

| | | TAC/LH230 | | TAC/LH206 | | TAC/LH230 |
|---|---|---|---|---|---|---|
| | TAC/LH230 | (10/90), | TAC/LH206 | (10/90), | TAC/LH230 | (30/70), |
| | (10/90), | ambient | (10/90), | ambient | (30/70), | ambient |
| | initial^{#} | 10M* | initial^{#} | 10M | initial^{#} | 10M |
| MMAD (μm) | 2.62 ± 0.03 | 1.65 ± 0.01 | 2.29 ± 0.29 | 1.74 ± 0.11 | 2.63 ± 0.21 | 1.79 ± 0.05 |
| GSD | 2.54 ± 0.02 | 3.21 ± 0.01 | 2.54 ± 0.66 | 1.94 ± 0.02 | 2.49 ± 0.11 | 2.46 ± 0.02 |
| FPF (%, | 51.12 ± 0.89 | 59.47 ± 0.68 | 57.24 ± 6.26 | 63.55 ± 1.69 | 57.07 ± 4.05 | 70.59 ± 1.76 |
| recovered dose) | | | | | | |
| FPF (%, | 53.98 ± 1.34 | 61.62 ± 1.06 | 61.06 ± 7.45 | 68.59 ± 0.53 | 61.11 ± 4.81 | 73.58 ± 2.46 |
| delivered dose) | | | | | | |
| EF (%) | 94.31 ± 0.69 | 96.99 ± 0.55 | 93.97 ± 2.56 | 92.64 ± 1.75 | 93.42 ± 0.75 | 95.94 ± 0.08 |

### 10. Analysis of Drug Loading on Composition Preparation

Furthermore, the effects of drug loading on the preparation of the pharmaceutical compositions were reviewed. In particular, two sets of compositions with different drug loading (1.67% and 6.67%) were carried out. Additionally, these compositions were prepared using a variety of different solvent systems and at different amounts of solid content. The specific composition of the systems is shown in Tables 21 & 22 below.

TABLE 21
Conditions Tested for preparation of a composition with a 1.67% w/w drug loading.

| | Drug | | Solid | | Processing |
|---|---|---|---|---|---|
| | loading | | content | | temperature |
| Formulation | (% w/w) | Lactose grade | (% w/v) | Solvent (v/v) | (° C.) |
| TAC-167-1 | 1.67 | Lactohale 206 | 5 | 1,4-dioxane | −100 |
| TAC-167-2 | 1.67 | Lactohale 206 | 5 | acetonitrile | −100 |
| TAC-167-3 | 1.67 | Lactohale 206 | 5 | acetonitrile/t-butanol (50/50) | −100 |
| TAC-167-4 | 1.67 | Lactohale 206 | 5 | acetonitrile/t-butanol (25/75) | −100 |
| TAC-167-5 | 1.67 | Lactohale 206 | 5 | acetonitrile/t-butanol (75/25) | −100 |
| TAC-167-6 | 1.67 | Lactohale 206 | 2.5 | acetonitrile/t-butanol (50/50) | −100 |
| TAC-167-7 | 1.67 | Lactohale 230 | 5 | acetonitrile/t-butanol (50/50) | −100 |
| TAC-167-8 | 1.67 | Respitose SV003 | 5 | acetonitrile | −100 |

TABLE 22
Conditions Tested for preparation of a composition with a 6.67% w/w drug loading

| | Drug | | Solid | | Processing |
|---|---|---|---|---|---|
| | loading | | content | | temperature |
| Formulation | (% w/w) | Lactose grade | (% w/v) | Solvent (v/v) | (° C.) |
| TAC-667-1 | 6.67 | Lactohale 206 | 5 | 1,4-dioxane | −100 |
| TAC-667-2 | 6.67 | Lactohale 206 | 5 | acetonitrile | −100 |
| TAC-667-3 | 6.67 | Lactohale 206 | 5 | t-butanol/acetonitrile (50/50) | −100 |
| TAC-667-4 | 6.67 | Lactohale 206 | 5 | t-butanol/acetonitrile (25/75) | −100 |
| TAC-667-5 | 6.67 | Lactohale 206 | 5 | t-butanol/acetonitrile (75/25) | −100 |
| TAC-667-6 | 6.67 | Lactohale 206 | 2.5 | t-butanol/acetonitrile (50/50) | −100 |
| TAC-667-7 | 6.67 | Lactohale 230 | 5 | t-butanol/acetonitrile (50/50) | −100 |
| TAC-667-8 | 6.67 | Respitose SV003 | 5 | acetonitrile | −100 |

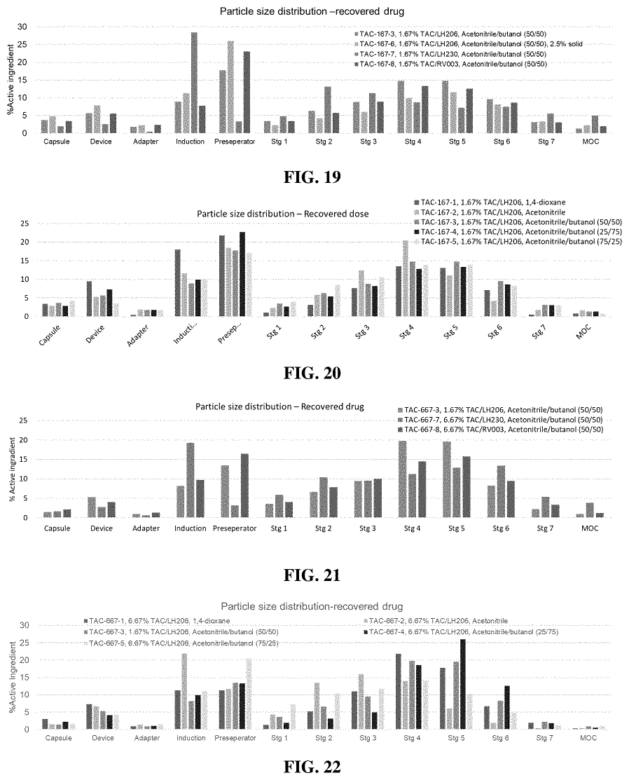

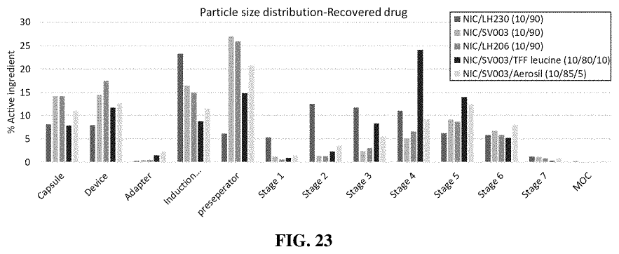

First, the particle size distribution and the relevant properties of 1.67% solid content compositions prepared with a mixture of acetonitrile and t-butanol with different lactose excipients. Each of these compositions showed properties such as MMAD and GSD as shown in Table 23 below. The distribution of these particles within the respiratory system is shown in FIG. 19. Similar analysis was done with varying the solvent system using to prepare the compositions. The properties of the resultant compositions are shown in Table 24 and FIG. 20.

Similar studies were carried out with a drug loading of 6.67% tacrolimus and are shown in Tables 25 & 26 as well as FIGS. 21 & 22.

TABLE 23
Resultant Compositions with 1.67% Tacrolimus Drug Loading Using Different Lactose Grades

| | TAC-167-3, | TAC-167-6, | 7TAC-167-7, | 7TAC-167-8, |
|---|---|---|---|---|
| | 1.67% TAC/LH206, | 1.67% TAC/LH206, | 1.67% TAC/LH230, | 1.67% TAC/SV003, |
| | 5% solid | 2.5% solid | 5% solid | 5% solid |
| | Acetonitrile/butanol | Acetonitrile/butanol | Acetonitrile/butanol | Acetonitrile/butanol |
| | (50/50) | (50/50) | (50/50) | (50/50) |
| MMAD (μm) | 1.82 | 1.57 | 2.48 | 1.87 |
| GSD | 2.48 | 2.62 | 2.46 | 2.44 |
| FPF (%, recovered dose) | 53.86 | 42.16 | 48.15 | 49.73 |
| FPF (%, delivered dose) | 59.41 | 48.26 | 50.46 | 54.66 |
| EF (%) | 90.65 | 87.36 | 95.42 | 90.98 |

TABLE 24
Resultant Compositions with 1.67% Tacrolimus Drug Loading
Using Different Solvent Systems to Prepare the Composition

| | TAC-167-1 | TAC-167-2, | TAC-167-3, | TAC-167-4, | TAC-167-5, |
|---|---|---|---|---|---|
| | 1.67% | 1.67% | 1.67% TAC/LH206, | 1.67% TAC/LH206, | 1.67% TAC/LH206, |
| | TAC/LH206, | TAC/LH206, | Acetonitrile/butanol | Acetonitrile/butanol | Acetonitrile/butanol |
| | 1,4-dioxane | Acetonitrile | (50/50) | (25/75) | (75/25) |
| MMAD (μm) | 1.81 | 2.26 | 1.82 | 1.78 | 2.08 |
| GSD | 1.99 | 1.89 | 2.48 | 2.43 | 2.51 |
| FPF (%, recovered dose) | 40.61 | 52.98 | 53.86 | 48.51 | 52.46 |
| FPF (%, delivered dose) | 46.61 | 57.68 | 59.41 | 53.99 | 56.96 |
| EF (%) | 87.14 | 91.85 | 90.65 | 89.86 | 92.11 |

TABLE 25
Resultant Compositions with 6.67% Tacrolimus
Drug Loading Using Different Lactose Grades

| | TAC-667-3, | 7TAC-667-7, | 7TAC-667-8, |
|---|---|---|---|
| | 6.67% TAC/LH206, | 6.67% TAC/LH230, | 6.67% TAC/SV003, |
| | 5% solid | 5% solid | 5% solid |
| | Acetonitrile/butanol | Acetonitrile/butanol | Acetonitrile/butanol |
| | (50/50) | (25/75) | (75/50) |
| MMAD (μm) | 1.88 | 1.75 | 1.90 |
| GSD | 2.89 | 3.25 | 2.59 |
| FPF (%, recovered dose) | 61.76 | 58.55 | 56.08 |
| FPF (%, delivered dose) | 66.22 | 61.23 | 59.79 |
| EF (%) | 93.27 | 96.63 | 93.80 |

TABLE 26
Resultant Compositions with 6.67% Tacrolimus Drug Loading
Using Different Solvent Systems to Prepare the Composition

| | | | TAC-667-3, | TAC-667-4, | TAC-667-5, |
|---|---|---|---|---|---|
| | TAC-667-1, | TAC-667-2, | 6.67% | 6.67% | 6.67% |
| | 6.67% | 6.67% | TAC/LH206, | TAC/LH206, | TAC/LH206, |
| | TAC/LH206, | TAC/LH206, | Acetonitrile/butanol | Acetonitrile/butanol | Acetonitrile/butanol |
| | 1,4-dioxane | Acetonitrile | (50/50) | (25/75) | (75/25) |
| MMAD (μm) | 1.95 | 3.35 | 1.88 | 1.49 | 2.28 |
| GSD | 1.90 | 1.88 | 2.89 | 1.85 | 2.51 |
| FPF (%, recovered dose) | 60.94 | 41.77 | 61.76 | 65.08 | 45.48 |
| FPF (%, delivered dose) | 67.95 | 45.60 | 66.22 | 69.08 | 48.31 |
| EF (%) | 89.68 | 91.61 | 93.27 | 93.69 | 94.13 |

### 10. Niclosamide Compositions

Similarly, the properties of niclosamide containing compositions were prepared as shown in Table 27 below. These compositions were prepared using similar methods to those described above for tacrolimus or voriconazole. These compositions were prepared with Lactohale LH206 and LH230 as well as Respitose SV003. Among these compositions, the compositions with Lactohale LH230 showed the best aerosol performance relative to the other grades of carrier. Furthermore, compositions were tested with the presence of additional excipients such as silica (Aerosil) or leucine. The addition of these secondary excipients generally improved the performance of the composition compared to those compositions without the secondary excipients. These compositions were tested as described above for MMAD, GSD, emitted dose or fraction, and fine particle fraction of both recovered or delivered dose. These data are shown in Table 28 below. These data were used to determine the distribution of a dose emitted by an inhaler into the lungs as shown in FIG. 27.

TABLE 27
Niclosamide Compositions

| | Drug | | | | % | Solid | | Processing |
|---|---|---|---|---|---|---|---|---|
| | loading | | | Secondary | Secondary | content | Solvent | temperature |
| Formulation | (% w/w) | Lactose grade | % Lactose | excipient | excipient | (% w/v) | (v/v) | (° C.) |
| NIC-10-1 | 10 | Lactohale 230 | 90 | — | 0 | 3 | 1,4-dioxane | −120 |
| NIC-10-2 | 10 | Respitose SV003 | 90 | — | 0 | 3 | 1,4-dioxane | −120 |
| NIC-10-3 | 10 | Lactohale 206 | 90 | — | 0 | 3 | 1,4-dioxane | −120 |
| NIC-10-4 | 10 | Respitose SV003 | 80 | TFF leucine | 10 | 3 | 1,4-dioxane | −120 |
| NIC-10-5 | 10 | Respitose SV003 | 85 | Aerosil 200 | 5 | 3 | 1,4-dioxane | −120 |

TABLE 28
Properties of the niclosamide compositions

| | NIC-10-1; | NIC-10-2; | NIC-10-3, | NIC-10-4, | NIC-10-4, |
|---|---|---|---|---|---|
| | NIC/LH230 | NIC/SV003 | NIC/LH206 | NIC/SV003/TFF | NIC/SV003/Aerosil |
| | 10/90 | 10/90 | 10/90 | leucine 10/80/10 | 200 10/85/5 |
| MMAD (μm) | 3.16 | 1.36 | 1.49 | 2.00 | 1.62 |
| GSD | 2.121 | 2.34 | 2.08 | 1.62 | 2.42 |
| FPF (%, recovered dose) | 38.92 | 25.04 | 25.39 | 52.51 | 37.29 |
| FPF (%, delivered dose) | 46.36 | 35.09 | 37.18 | 65.31 | 48.87 |
| EF (%) | 83.97 | 71.35 | 68.29 | 80.40 | 76.29 |

### D. Discussion

Powders made using the suspension based TFF process are aerosolizable and homogenous. The ordered mixture containing the drug and inhalation-grade LAC can be prepared using the suspension based TFF process. The aerosol performance and homogeneity of powders made using the suspension based TFF process was compared to those of powders made using conventional blending. Our results indicate that powders made using the suspension based TFF process exhibited better aerosol performance and more uniform powder than the same formulation compositions made using conventional blending.

The homogeneity of the drug in dry powders made using the suspension based process is possibly related to the degree of deagglomeration of the drug and its carrier. TFF of the suspensions resulted in the agglomeration of drug particles on a carrier surface. Despite the various particle morphologies, both the nanoaggregates of VCZ and the TAC nanostructure brittle matrix can closely adhere to the surface of the LAC carrier. In the formulations containing high drug-carrier ratios and smaller size of LAC, LAC carriers were also covered by the brittle matrix of the drug. Additionally, the ultra-rapid freezing rate of TFF can possibly minimize segregation during processing, which provides the benefit over other ordered mixing approaches.

The degree of deagglomeration of the ordered mixture was determined by critical primary pressure (Jaffari et al., 2013). The critical primary pressure represents the dispersing pressure that can overcome the interparticulate forces that hold the ordered mixture powder together (Jaffari et al., 2013). As shown in FIGS. 9 and 16, TFF neat VCZ and neat TAC generally required higher pressures than TFF neat LAC, indicating that neat LAC is easier to deagglomerate than the drugs in the brittle matrix. As might be expected, the combination of the brittle matrix of the drug and LAC resulted in higher CPP than neat LAC. Additionally, the CPPs of powders made using the suspension based TFF process were higher than those of powders made using conventional blending. This indicates that the degree of deagglomeration of powders made using the suspension based TFF process is lower than powders made using conventional blending, which means the powders made using the suspension based TFF process need higher pressure to overcome the interparticulate force between the drugs can carrier.

The homogeneity and deagglomeration of the ordered mixture upon aerosolization are dependent on the cohesive (drug-drug) and the adhesive (drug-carrier) forces (Begat et al., 2004). Several studies have reported that the interaction between the drug and excipient host particles can reduce the risk of segregation (Lai et al., 1981; Wal Yip and Hersey, 1977; Crooks and Ho, 1976; Thiel and Stephenson, 1982). The extent and intensity of the interparticulate forces affect the degree of segregation and subsequently affect the homogeneity of the ordered mixture (Chaudhuri et al., 2006). We hypothesized that the low degree of deagglomeration in powders made using suspension based TFF process indicates that the interparticulate forces between the drug and carrier are stronger than those in the powder made using conventional blending. This helps minimize the segregation problem, thus improving the homogeneity of the ordered mixture.

Strong agglomeration is typically undesirable for carrier-based formulations, because it affects the dispersibility and detachment of the drug from its carrier (de Boer et al., 2012). However, our results demonstrate that the aerosolization of the TFF ordered mixture was not correlated with the degree of deagglomeration measured by laser diffraction. Although powders made using the suspension based TFF process exhibited less deagglomeration than the powder made using conventional blending, the aerosol performance of both TAC and VCZ in powders made using the suspension based TFF process was higher than the performance of powders made using conventional blending (FIGS. 6, 7, 13, and 14). This is likely related to various dispersing mechanisms between powders made using the suspension based TFF process versus conventional blending. Although the ordered mixture mostly contained LAC carrier, the surface area of the powders made using the suspension based TFF process was larger than the unprocessed powders and the powder made using conventional blending (FIGS. 4 and 12). Porous particles have less contact area and less interparticulate force (Weers, 2000), which can be sheared apart by the upon aerosolization. In contrast, flat surface of jet milled TAC and VCZ has relatively larger contact area and stronger interparticulate force (Hinds, 1999), which can minimize the drug detachment from a carrier.

Carrier particle size and drug loading affects the aerosolization of powders made using the suspension based TFF process. The influence of carrier particle size on drug aerosolization performance has been previously studied in the literature (Grasmeijer et al., 2015; Peng et al., 2016). Despite the inconsistent trends in the effect of carrier size on aerosol performance reported in the literatures (Grasmeijer et al., 2015; Peng et al., 2016), larger carrier size resulted in an increase in the FPF of TAC and VCZ. Both drug cases showed that TFF formulations containing Lactohale® LH300 exhibited lower FPF and EF than other TFF formulations containing larger size of LAC. Lactohale® LH300 is a very fine and micronized LAC grade with a Dv50 below 5 μm (DFE Pharma, 2020). Due to its very small particle size, the cohesivity of Lactohale® LH300 is higher than other grades, which allows more drug to attach and agglomerate. This is consistent with Guenette's study reporting that the ultrafine LAC particles are highly cohesive, leading to an increase in powder aggregation (Grasmeijer et al., 2015).

In addition to a very fine grade of LAC, three different LAC grades were used in this study. Lactohale® LH230 is fine-milled LAC, while Lactohale® LH206 is a coarse-milled LAC that contains no fine LACparticles (DFE Pharma, 2020). Repitose® SV003 differs from Lactohale, since it is composed of fine-sieved LAC crystals with a narrow particle size distribution. Both drug cases showed coarse LAC can improve the aerosol performance. In the case of TAC, the FPF of the formulation containing Lactohale® LH206 was significantly higher than other LAC grades. Similarly, VCZ formulations containing Respitose® SV003 and Lactohale® 206 exhibited significantly smaller MMAD and higher FPF compared to fine LAC. A trend of increasing aerosol performance by carrier size is consistent with the findings from several studies. It has been reported that the larger size of LAC can increase the collisions forces between the carrier particles, and between the carrier particles and the inhaler wall, which increase the momentum transfer and subsequently increase drug detachment from the carrier (Kaialy et al., 2012; Donovan and Smyth, 2010; Donovan et al., 2012; Ooi et al., 2011).

Moreover, drug loading in powders made using the suspension based TFF process suspension based TFF process also affected the aerosol performance of both TAC and VCZ. Both drugs exhibited the same trends. An increase in drug loading below 10% resulted in an increase in aerosol performance. The aerosol performance was not influenced by the drug content when the drug content exceeded 10%. This finding agrees with the literature reporting that FPF increases as the drug loading is increased, after it reaches a critical threshold due to the saturation of active sites on the LACcarrier surface (Young et al., 2005; Du et al., 2017). Since the surfaces of LAC are heterogenous, containing pits and crevices as well as various crystal facets, the surface will contain both low- and high-adhesion sties (Young et al., 2011). The drug is preferentially bind to the high adhesion sites (active sites) first, followed by the lower adhesion sites. At the critical threshold, the binding capacity of active sites reaches its maximum. A further increase in drug content will allow the drug to bind to the intermediate adhesion sites, thus increasing the ease of deagglomeration. However, at the certain point, a further increase in drug content will allow the the drug particles to bind to the remaining low adhesive sites and form a monolayer on the carrier, which results in constant fine particle fraction. It has also been reported that the point that exhibit constant fine particle fraction depends on the carrier size (Young et al., 2005; de Boer et al., 2005; Dickhoff et al., 2003). Since only Lactohale® LH230 was used to investigate the effect of drug loading in our study, the same threshold (i.e., 10% drug loading) observed in both drug cases relates to the binding capacity of Lactohale® LH230.

Carrier size and drug loading appear to have a little effect on the homogeneity of TFF ordered mixtures. Both drug cases showed high variation in blend uniformity in the formulations containing Lactohale® LH206; however, no clear trend was observed in other carrier sizes. We hypothesized that the content uniformity may be diminished by some of the TAC brittle matrix and the nanoaggregates of VCZ that are not attached to the surface of the carrier. Interestingly, drug loading did not significantly affect the homogeneity of the TFF ordered mixtures. The TFF VCZ formulation containing 1% drug loading showed more variation than other ratios, but there is no significant trend over the entire range of drug loading.

Secondary excipients affect drug aerosolization, but the effect varies based on the dispersing mechanism of different particle morphology. In our study, secondary excipients were added to the ordered mixture. PVP K25 was dissolved and mixed with the drug in the solvent before dispersing the LAC carrier in the antisolvent. Our results show that the addition of PVP K25 did not improve the aerosol performance of TAC or VCZ. This observation agrees with Traini's study. It was reported that that PVP coating on the surface of the LAC carrier increased drug-carrier adhesion, thus decreasing aerosol performance (Traini et al., 2012). We hypothesized that some parts of PVP K25 can form a nanostructure brittle matrix with a drug, while other parts of PVP may coat the surface of the LAC. As a result, it is possible that the adhesion of VCZ nanoaggregates or TAC nanostructured brittle matrix on coated LAC may be stronger than uncoated LAC, thus minimizing drug detachment from the carrier.

The presence of leucine in the formulation appears to improve the aerosol performance of VCZ, but it did not affect the aerosol performance of TAC. Engineered particles have been reported as a force control agent that can modify the interparticulate forces (Grasmeijer et al., 2015). Due to the difference in particle morphology and the physical properties between VCZ nanoaggregates and TAC nanostructured brittle matrix, the dispersing mechanism of the powders are different, which governs the impact of the engineered leucine on aerosolization.

The TAC nanostructured brittle matrix formed on the LAC carrier and exhibited a large specific surface area. Our result demonstrates that the addition of engineered leucine did not improve the aerosol performance of TAC. It was reported that a particle with a highly porous surface has a shorter interparticle separation distance, less contact area, and weaker interparticle cohesive forces (Weers, 2000). Therefore, it is possible that the surface energy of the TAC brittle matrix is sufficiently low to aerosolize without the addition of a surface modifier.

In contrast, an improvement in the aerosol performance of VCZ is likely attributable to the change of cohesive and adhesive forces by the addition of a surface modifier. We hypothesized that TFF leucine and jet-milled leucine may attach to both VCZ nanoaggregates and the surface of the LAC carrier, which can subsequently minimize both the cohesive forces between the drug particles and the adhesive forces between the drug and its LAC carrier. It was reported that VCZ nanoaggregates are required a small amount of surface texture-modifying excipient (Moon et al., 2019). Due to the flat surface of VCZ nanoaggregates, the contact area is large, thus particle cohesion is high (Duddu et al., 2002), which makes the drug itself difficult to aerosolize. The aerosol performance of VCZ can be improved by the addition of a small amount of mannitol. Mannitol particles adhere to the surface of VCZ nanoaggregates and function as a surface texture modifier (Moon et al., 2019). Similar to our cases, leucine can minimize the contact area and distance between particles when attached to VCZ nanoaggregates and the LAC carrier (Paajanen et al., 2009; Mangal et al., 2019). This subsequently decreases the van der Waals forces between the particles (Hinds, 1999), which is the main adhesive force that affects aerosol performance (Hickey, 1994). Therefore, the aerosol performance of the TFF VCZ ordered mixture can be optimized by adding engineered leucine.

This study has confirmed that TFF is a feasible single-step method to prepare an ordered mixture, especially those intended for dry powder inhalation. The suspension based TFF process creates niclosamide compositions, voriconazole nanoaggregates, and tacrolimus nanostructured brittle matrices in which the drug agglomerates with the LAC carrier strongly. This provides the benefit of a reduced risk of segregation. The lower degree of deagglomeration did not affect the aerosol performance of the TFF ordered mixture. The aerosol performance of the TFF ordered mixture can be optimized by varying the drug loading and carrier size and by adding engineered leucine. The homogeneity of powders made using the suspension based TFF process was in the acceptable range and was not significantly influenced by carrier size, drug loading, or the presence of a secondary excipient.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Adi et al., Journal of Pharmaceutical Sciences 2008, 97 (8), 3140-3152.
Begat et al., KONA Powder and Particle Journal 2005, 23, 109-121.
Begat et al., Pharmaceutical Research 2004, 21 (10), 1826-1833.
Carpenter et al., Pharm Res 1997, 14 (8), 969-75.
Chan and Chew, Adv Drug Deliv Rev 2003, 55 (7), 793-805.
Chaudhuri et al., Powder Technology 2006, 165 (2), 105-114.
Crooks and Ho, Powder Technology 1976, 14 (1), 161-167.
Crowder et al., Pharmaceutical Research 2002, 19 (3), 239-245.
de Boer et al., Adv Drug Deliv Rev 2012, 64 (3), 257-74.
de Boer et al., Int J Pharm 2005, 294 (1-2), 173-84.
DFE Pharma, Excipients, Dry Powder Inhalation, Inhalation. https://www.dfepharma.com.br/Excipients/Expertise/Dry-Powder-Inhalation/Inhalation (accessed Mar. 23, 2020).
Dickhoff et al., European Journal of Pharmaceutics and Biopharmaceutics 2003, 56 (2), 291-302.
Donovan et al., Int J Pharm 2010, 402 (1-2), 1-9.
Donovan et al., J Pharm Sci 2012, 101 (3), 1097-107.
Du et al., Journal of Pharmaceutical Sciences 2017, 106 (1), 366-376.
Duddu et al., Pharmaceutical research 2002, 19 (5), 689-695.
Grasmeijer et al., Curr Pharm Des 2015, 21 (40), 5900-14.
Grasmeijer et al., PLoS One 2013, 8 (7), e69263.
Griffin, Expert review of vaccines 2014, 13 (6), 751-9.
Hassan et al., Int J Pharm 2010, 386 (1-2), 6-14.
Hickey, Pharm. Technol. 1994, 18, 58-82.
Hinds, Aerosol technology: properties, behavior, and measurement of airborne particles. John Wiley & Sons: 1999.
Jaffari et al., Int J Pharm 2013, 447 (1-2), 124-31.
Jones et al., Int J Pharm 2010, 391 (1-2), 137-47.
Kaialy et al., Pharmaceutical Research 2013, 31 (1), 60-76.
Kaialy et al., Powder Technology 2012, 227, 74-85.
Kaialy, Int J Pharm 2016, 503 (1-2), 262-76.
Kamer et al., Powder Technology 2014, 264, 544-549.
Kristin et al., Current Pharmaceutical Design 2016, 22 (17), 2501-2521.
Lai et al., Powder Technology 1981, 28 (1), 17-23.
Le et al., AAPS PharmSciTech 2012, 13 (2), 477-84.
LeWitt et al., J Aerosol Med Pulm Drug Deliv 2018, 31 (3), 155-161.
Longest et al., Pharm Res 2017, 34 (10), 2049-2065.
Mangal et al., Int J Pharm 2019, 568, 118504.
Moon et al., Mol Pharm 2019, 16 (5), 1799-1812.
Ooi et al., Int J Pharm 2011, 413 (1-2), 1-9.
Overhoff et al., Journal of Drug Delivery Science and Technology 2009, 19 (2), 89-98.
Paajanen et al., Powder Technology 2009, 192 (1), 6-11.
Peng et al., Acta Pharm Sin B 2016, 6 (4), 308-18.
Pfutzner and Forst, Expert Opin Drug Deliv 2005, 2 (6), 1097-106.
Poux et al., Powder Technology 1991, 68 (3), 213-234.
Prime et al., Advanced Drug Delivery Reviews 1997, 26 (1), 51-58.
Pu et al., J Pharm Sci 2009, 98 (7), 2412-21.
Sarkar et al., J Pharm Sci 2017, 106 (1), 129-139.
Silveira et al., Antiviral therapy 2016, 21 (1), 71-4.
Singh et al., Journal of Aerosol Medicine and Pulmonary Drug Delivery 2015, 28 (4), 254-267.
Staniforth, Relation between mixing time and segregation of ordered mixes. 1981.
Steckel et al., Eur J Pharm Biopharm 2004, 57 (3), 495-505.
Tan et al., In Pharmaceutical Inhalation Aerosol Technology, Third ed.; J. Hickey, A.; Rocha, S. R. P. d., Eds. CRC Press Taylor&Francis group: Florida, USA, 2019.
Tee et al., International Journal of Pharmaceutics 2000, 208 (1), 111-123.
Thiel et al., Powder Technology 1982, 31 (1), 45-50.
Traini et al., Int J Pharm 2012, 438 (1-2), 150-9.
Wal Yip, C.; Hersey, J. A., Perfect powder mixtures. Powder Technology 1977, 16 (2), 189-192.
Wang et al., AAPS PharmSciTech 2014, 15 (4), 981-93.
Watts et al., Pharm Res 2013, 30 (3), 813-25.
Weers, Innov Pharm Technol 2000, 1, 111-116.
Wyttenbach and Kuentz, Eur J Pharm Biopharm 2017, 112, 204-208.
Young et al., Int J Pharm 2011, 416 (1), 129-35.
Young et al., International Journal of Pharmaceutics 2005, 296 (1-2), 26-33.
Young et al., Journal of Pharmaceutical Sciences 2007, 96 (5), 1331-1341.
Zeng et al., International Journal of Pharmaceutics 1999, 182 (2), 133-144.
Zeng et al., International journal of pharmaceutics 2000, 197 (1-2), 41-52.
Zhou and Morton, Adv Drug Deliv Rev 2012, 64 (3), 275-84.

1. A method of preparing a pharmaceutical composition comprising:
(A) obtaining a solution of an active pharmaceutical ingredient in a solvent;
(B) adding a carrier to the mixture to obtain a dispersion;
(C) subjecting the dispersion to a reduced temperature surface such that the dispersion freezes to obtain a frozen dispersion; and
(D) subjecting the frozen dispersion to a drying process to obtain a pharmaceutical composition;
wherein the pharmaceutical composition contains one or more particles wherein the active pharmaceutical ingredient has been deposited on the surface of the carrier.

2. The method of claim 1, wherein the dispersion further comprises a further excipient.

3. The method of claim 2, wherein the excipient is an amino acid.

4-8. (canceled)

9. The method of claim 1, wherein the carrier is a sugar or sugar alcohol.

10-11. (canceled)

12. The method of claim 1, wherein the carrier is sparingly soluble in the solvent.

13-15. (canceled)

16. The method of claim 1, wherein the dispersion is a suspension.

17-28. (canceled)

29. The method of claim 1, wherein the pharmaceutical composition comprises from about 50% w/w to about 99% w/w of the carrier.

31. (canceled)

32. The method of claim 1, wherein the mixture further comprises a pharmaceutically acceptable polymer.

33-40. (canceled)

41. The method of claim 1, wherein the solvent is an organic solvent.

42. The method of claim 41, wherein the organic solvent is a polar aprotic solvent.

43-46. (canceled)

47. The method of claim 1, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

48. The method of claim 47, wherein the active pharmaceutical ingredient is antifungal agent, an immunomodulating drug, or an anthelmintic drug.

49-70. (canceled)

71. The method of claim 1, wherein the method further comprises using a surface that has been cooled to a first reduced temperature.

72. The method of claim 71, wherein the first reduced temperature is from about 25° C. to about −190° C.

73-74. (canceled)

75. The method of claim 1, wherein the surface rotates at a speed.

76-104. (canceled)

105. The method of claim 1, wherein the pharmaceutical composition comprises one or more particles of the active pharmaceutical ingredient and the carrier are agglomerated.

106-109. (canceled)

110. The method of claim 1,
wherein the pharmaceutical composition has a specific surface area of greater than 2 m²/g.

111-114. (canceled)

115. The method of claim 1, wherein the pharmaceutical composition has a specific surface area that is 50% greater than the specific surface area of the carrier.

116-140. (canceled)

141. The method of claim 1, wherein relative standard deviation of the homogeneity of the pharmaceutical composition is 50% less than the relative standard deviation of the homogeneity of a pharmaceutical composition prepared using other means.

142-169. (canceled)

170. A pharmaceutical composition comprising:
(A) an active pharmaceutical ingredient;
(B) a carrier;
wherein the pharmaceutical composition contains one or more particles wherein the active pharmaceutical ingredient has been deposited on the surface of the carrier, the pharmaceutical composition comprises both the active pharmaceutical ingredient and the carrier in a single particle, and the pharmaceutical composition has a specific surface area that is 50% greater than the specific surface area of the carrier.

171. The pharmaceutical composition of claim 170, wherein the pharmaceutical composition further comprises a further excipient.

172-177. (canceled)

178. The pharmaceutical composition of claim 170, wherein the carrier is a sugar or sugar alcohol.

179-192. (canceled)

193. The pharmaceutical composition of claim 170, wherein the pharmaceutical composition comprises from about 50% w/w to about 99% w/w of the carrier.

194-195. (canceled)

196. The pharmaceutical composition of claim 170, wherein the pharmaceutical composition further comprises a pharmaceutically acceptable polymer.

197-204. (canceled)

205. The pharmaceutical composition of claim 170, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

206. The pharmaceutical composition of claim 205, wherein the active pharmaceutical ingredient is antifungal agent, an immunomodulating drug, or an anthelmintic drug.

207-235. (canceled)

236. The pharmaceutical composition of claim 170, wherein the pharmaceutical composition comprises one or more particles of the active pharmaceutical ingredient and the carrier are agglomerated.

237-245. (canceled)

246. The pharmaceutical composition of claim 170, wherein the pharmaceutical composition has a specific surface area that is 75% greater than the specific surface area of the carrier.

247-270. (canceled)

271. The pharmaceutical composition of claim 170, wherein relative standard deviation of the homogeneity of the pharmaceutical composition is 50% less than the relative standard deviation of the homogeneity of a pharmaceutical composition prepared using other means.

272-299. (canceled)

300. A pharmaceutical composition comprising:
(A) an active pharmaceutical ingredient, wherein the active pharmaceutical ingredient is antifungal agent, anthelminthic agent, or immunomodulating compound; and
(B) a carrier, wherein the carrier is a sugar;
wherein the pharmaceutical composition contains one or more particles wherein the active pharmaceutical ingredient has been deposited on the surface of the carrier, the pharmaceutical compositions comprises both the active pharmaceutical ingredient and the carrier in a single particle, and the pharmaceutical composition has a specific surface area that is 50% greater than the specific surface area of the carrier.

301-307. (canceled)

308. The pharmaceutical composition of claim 300, wherein the immunomodulating is tacrolimus.

309. The pharmaceutical composition of claim 300, wherein the antifungal is voriconazole

310. The pharmaceutical composition of claim 300, wherein the antihelminthic is niclosamide.
